(19) United States
(12) Patent Application Publication
(10) Pub. No.: US 2004/0014634 A1
(43) Pub. Date: Jan. 22, 2004

(54) Thieno[2,3-d]pyrimidinediones and their use as pharmaceuticals

(76) Inventors: John Bantick, Loughborough (GB); Anthony Ingall, Loughborough (GB); Matthew Perry, Loughborough (GB); Rachel Reynolds, Loughborough (GB)

Correspondence Address: MORGAN LEWIS & BOCKIUS LLP, 1111 PENNSYLVANIA AVENUE NW, WASHINGTON, DC, 20004, US

(21) Appl. No.: 10/275,344

(22) PCT Filed: Apr. 26, 2001
(86) PCT No.: PCT/SE01/00907

(30) Foreign Application Priority Data
May 4, 2000 (GB) 0010657.5

(51) Int. Cl. A61K 31/00

(52) U.S. Cl. 514/1

(57) ABSTRACT

The invention provides thieno[2,3-d]pyrimidinediones of general formula (I), wherein R, R¹, R² and R³ are as defined in the specification, processes for their production, pharmaceutical compositions containing them and their use in therapy.

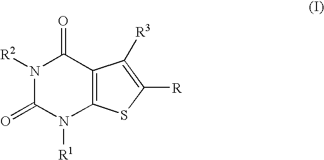

The present invention relates to thieno[2,3-d]pyrimidinediones, processes for their preparation, pharmaceutical compositions containing them and their use in therapy.

In accordance with the present invention, there is provided a compound of general formula

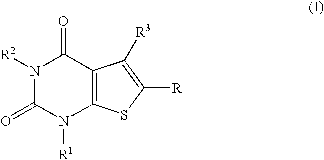

wherein:

R is —C(O)Ar¹, —C(R⁴)(R⁵)Ar¹ or Ar³;

Ar¹ represents a 5- to 10-membered aromatic ring system wherein up to 3 ring atoms may be heteroatoms independently selected from nitrogen, oxygen and sulphur, the ring system being optionally substituted by one or more substituents independently selected from C₁₋₄ alkyl, C₁₋₄ alkoxy, halogen, trifluoromethyl, oxo, nitro, cyano, NR⁶R⁷ and —CH₂NR⁸R⁹;

R¹ and R² each independently represent a hydrogen atom, C₁₋₆ alkyl, C₃₋₆ alkenyl, CH₂C₃₋₅ cycloalkyl or C₃₋₆ cycloalkyl;

R³ represents a group X—Ar²;

X represents a group S(O)ₙ, C(O) or CH(OH);

n is 0, 1 or 2;

Ar² represents a 5- or 6-membered aromatic ring wherein up to 4 ring atoms may be heteroatoms independently selected from nitrogen, oxygen and sulphur, the ring being optionally substituted by one or more substituents independently selected from C₁₋₄ alkyl, C₁₋₄ alkoxy, C₁₋₄ alkylthio, halogen, trifluoromethyl, oxo, hydroxyl, amino (NH₂), nitro, cyano and benzyl;

R⁴ represents a hydrogen atom or C₁₋₄ allyl (e.g. methyl, ethyl, n-propyl or n-butyl);

R⁵ represents a hydrogen atom or hydroxyl group;

R⁶ and R⁷ each independently represent a hydrogen atom or C₁₋₄ alkyl, or together with the nitrogen atom to which they are attached form a 5- to 7-membered saturated heterocyclic ring;

R⁸ and R⁹ each independently represent a hydrogen atom or C₁₋₄ alkyl, or together with the nitrogen atom to which they are attached form a 5- to 7-membered saturated heterocyclic ring; and

Ar³ represents acenaphthenyl, indanyl or fluorenyl, each of which may be optionally substituted by one or more substituents independently selected from C₁₋₄ alkyl, C₁₋₄ alkoxy, halogen or trifluoromethyl;

with the proviso that when X represents S(O)ₙ, then Ar² does not represent pyridyl or thienyl;

or a pharmaceutically acceptable salt or solvate thereof.

In the present specification, unless otherwise indicated, an allyl or alkenyl group or an alkyl or alkenyl moiety in a substituent group may be linear or branched. It will be appreciated that when R represents a group —C(R⁴)(R⁵)Ar¹, R⁵ may represent a hydroxyl group only when Ar¹ is bonded to —C(R⁴)(R⁵) through a carbon atom and not a heteroatom. Furthermore, it should be understood that when R represents a group —C(O)Ar¹, Ar¹ is bonded through a carbon atom and not a heteroatom to the moiety —C(O).

R preferably represents —C(R⁴)(R⁵)Ar¹. R⁴ and R⁵ preferably both represent a hydrogen atom.

Ar¹ represents a 5- to 10-membered aromatic ring system wherein up to 3 ring atoms may be heteroatoms independently selected from nitrogen, oxygen and sulphur, the ring system being optionally substituted by one or more (e.g. one, two, three or four) substituents independently selected from C₁₋₄ alkyl (e.g. methyl, ethyl, n-propyl or n-butyl), C₁₋₄ alkoxy (e.g. methoxy, ethoxy, n-propoxy or n-butoxy), halogen (e.g. fluorine, chlorine, bromine or iodine), trifluoromethyl, oxo, nitro, cyano, NR⁶R⁷ and —CH₂NR⁸R⁹. Preferred substituents to use are C₁₋₄ alkyl, halogen and trifluoromethyl.

The aromatic ring system may be monocyclic or polycyclic (e.g. bicyclic), examples of which include phenyl, naphthyl, pyrazolyl, thienyl, oxazolyl, imidazolyl, pyridinyl, pyridopyrrolyl, benzimidazolyl, indazolyl, benzothiazolyl, benzoxazolyl, thiazolyl and benzotriazolyl. Preferably, the aromatic ring system is monocyclic and 5- or 6-membered, especially phenyl.

R¹ and R² each independently represent a hydrogen atom, C₁₋₆, preferably C₁₋₄, alkyl (e.g. methyl, ethyl, n-propyl, 1-methylethyl, n-butyl, 2-methylpropyl, n-pentyl or n-hexyl), C₃₋₆, preferably C₃₋₄, alkenyl (e.g. 1-propenyl, 1-butenyl, 1-pentenyl or 1-hexenyl), CH₂C₃₋₅ cycloalkyl (cyclopropylmethyl, cyclobutylmethyl or cyclopentylmethyl) or C₃₋₆, preferably C₅₋₆, cycloalkyl (cyclopropyl, cyclobutyl, cyclopentyl or cyclohexyl).

Most preferably R¹ and R² each independently represent a C₁₋₄ alkyl group.

R⁶ and R⁷ each independently represent a hydrogen atom or C₁₋₄ alkyl (e.g. methyl, ethyl, n-propyl or n-butyl), or together with the nitrogen atom to which they are attached form a 5- to 7-membered saturated heterocyclic ring.

R⁸ and R⁹ each independently represent a hydrogen atom or C₁₋₄ alkyl (e.g. methyl, ethyl, n-propyl or n-butyl), or together with the nitrogen atom to which they are attached form a 5- to 7-membered saturated heterocyclic ring.

Ar² represents a 5- or 6-membered aromatic ring wherein up to 4 ring atoms may be heteroatoms independently selected from nitrogen, oxygen and sulphur, the ring being optionally substituted by one or more (e.g. one, two or three) substituents independently selected from C₁₋₄ alkyl (e.g. methyl, ethyl, n-propyl or n-butyl), C₁₋₄ alkoxy (e.g. methoxy, ethoxy, n-propoxy or n-butoxy), C₁₋₄ alkylthio (e.g. methylthio, ethylthio, n-propylthio or n-butylthio), halogen (e.g. fluorine, chlorine, bromine or iodine), trifluoromethyl, oxo, hydroxyl, amino, nitro, cyano and benzyl. A preferred substituent is C₁₋₄ alkyl. Examples of aromatic rings that can be used include furyl, pyridyl, thienyl, pyrrolyl, thiazolyl, thiadiazolyl, oxazolyl, imidazolyl, triazolyl and tetrazolyl.

Ar³ represents acenaphthenyl, indanyl or fluorenyl, each of which may be optionally substituted by one or more (e.g. one, two or three) substituents independently selected from C₁₋₄ alkyl (e.g. methyl, ethyl, n-propyl or n-butyl), C₁₋₄ alkoxy (e.g. methoxy, ethoxy, n-propoxy or n-butoxy), halogen (e.g. fluorine, chlorine, bromine or iodine) and trifluoromethyl.

Preferred compounds of the invention include:

5-[3-Furyl(hydroxy)methyl]-1-isobutyl-3-methyl-6-[2-(trifluoromethyl)benzyl]thieno[2,3-d]pyrimidine-2,4(1H,3H)-dione,

5-[2-Furyl(hydroxy)methyl]-1-isobutyl-3-methyl-6-[2-(trifluoromethyl)benzyl]-thieno[2,3-d]pyrimidine-2,4(1H,3H)-dione,

5-[Hydroxy(5-methyl-2-thienyl)methyl]-1-isobutyl-3-methyl-6-[2-(trifluoromethyl)benzyl]-thieno[2,3-d]pyrimidine-2,4(1H,3H)-dione,

5-[Hydroxy(3-pyridinyl)methyl]-1-isobutyl-3-methyl-6-[2-(trifluoromethyl)benzyl]-thieno[2,3-d]pyrimidine-2,4(1H,3H)-dione,

5-[Hydroxy(2-thienyl)methyl]-1-isobutyl-3-methyl-6-[2-(trifluoromethyl)benzyl]thieno[2,3-d]pyrimidine-2,4(1H,3H)-dione,

5-[Hydroxy(3-thienyl)methyl]-1-isobutyl-3-methyl-6-[2-(trifluoromethyl)benzyl]-thieno[2,3-d]pyrimidine-2,4(1H,3H)-dione,

1-Isobutyl-3-methyl-5-[(1-methyl-1H-pyrrol-2-yl)carbonyl]-6-[2-(trifluoromethyl)benzyl]-thieno[2,3-d]pyrimidine-2,4(1H,3H)-dione,

1-Isobutyl-3-methyl-5-(3-thienylcarbonyl)-6-[2-(trifluoromethyl)benzyl]-thieno[2,3-d]pyrimidine-2,4(1H,3H)-dione,

1-Isobutyl-3-methyl-5-[(3-methyl-2-pyridinyl)carbonyl]-6-[2-(trifluoromethyl)benzyl]-thieno[2,3-d]pyrimidine-2,4(1H,3H)-dione,

1-Isobutyl-3-methyl-5-(3-pyridinylcarbonyl)-6-[2-(trifluoromethyl)benzyl]-thieno[2,3-d]pyrimidine-2,4(1H,3H)-dione,

1-Isobutyl-3-methyl-5-(2-thienylcarbonyl)-6-[2-(trifluoromethyl)benzyl]-thieno[2,3-d]pyrimidine-2,4(1H,3H)-dione,

3-Methyl-1-(2-methylpropyl)-5-(1H-1,2,4-triazol-3-ylthio)-6-[2-(trifluoromethyl)benzoyl]-thieno[2,3 -d]pyrimidine-2,4(1H,3H)-dione,

3-Methyl-1-(2-methylpropyl)-5-(1H-1,2,4-triazol-3-ylthio)-6-[[2-(trifluoromethyl)phenyl]methyl]-thieno[2,3-d]pyrimidine-2,4(1H,3H)-dione,

3-Methyl-1-(2-methylpropyl)-5-(1H-1,2,4-triazol-3-ylsulfonyl)-6-[[2-(trifluoromethyl)phenyl]methyl]-thieno[2,3-d]pyrimidine-2,4(1H,3H)-dione,

3-Methyl-1-(2-methylpropyl)-5-(2-thiazolylthio)-6-[2-(trifluoromethyl)benzoyl]-thieno[2,3-d]pyrimidine-2,4(1H,3H)-dione,

3-Methyl-1-(2-methylpropyl)-5-[[5-(methylthio)- 1,3,4-thiadiazol-2-yl]thio]-6-[2-(trifluoromethyl)benzoyl]-thieno[2,3-d]pyrimidine-2,4(1H,3H)-dione,

3-Methyl-5-[(4-methyl-2-oxazolyl)thio]-1-(2-methylpropyl)-6-[2-(trifluoromethyl)benzoyl]-thieno[2,3-d]pyrimidine-2,4(1H,3H)-dione,

3-Methyl-1-(2-methylpropyl)-5-[(5-methyl-1H-1,2,4-triazol-3-yl)thio]-6-[2-(trifluoromethyl)benzoyl]-thieno[2,3-d]pyrimidine-2,4(1H,3H)-dione,

3-Methyl-1-(2-methylpropyl)-5-[(5-hydroxy-4-(1-methylethyl)-1H-1,2,4-triazol-3-yl)thio]-6-[2-(trifluoromethyl)benzoyl]-thieno[2,3-d]pyrimidine-2,4(1H,3H)-dione,

3-Methyl-1-(2-methylpropyl)-5-[(1-methyl-1H-tetrazol-5-yl)thio]-6-[2-(trifluoromethyl)benzoyl]- thieno[2,3-d]pyrimidine-2,4(1H,3H)-dione,

3-Methyl-1-(2-methylpropyl)-5-[(5-methyl-1,3,4-thiadiazol-2-yl)thio]-6-[2-(trifluoromethyl)benzoyl]-thieno[2,3-d]pyrimidine-2,4(1H,3H)-dione,

5-(1H-Imidazol-2-ylthio)-3-methyl-1-(2-methylpropyl)-6-[2-(trifluoromethyl)benzoyl]-thieno[2,3-d]pyrimidine-2,4(1H,3H)-dione,

5-[[4-(1,1-Dimethylethyl)-4H-1,2,4-triazol-3-yl]thio]-3-methyl-1-(2-methylpropyl)-6-[2-(trifluoromethyl)benzoyl]-thieno[2,3-d]pyrimidine-2,4(1H,3H)-dione,

3-Methyl-1-(2-methylpropyl)-5-(2-thienylthio)-6-[2-(trifluoromethyl)benzoyl]-thieno[2,3-d]pyrimidine-2,4(1H,3H)-dione,

5-[(5-Amino-1,3,4-thiadiazol-2-yl)thio]-3-methyl-1-(2-methylpropyl)-6-[2-(trifluoromethyl)benzoyl]-thieno[2,3-d]pyrimidine-2,4(1H,3H)-dione,

3-Methyl-1-(2-methylpropyl)-5-(1,3,4-thiadiazol-2-ylthio)-6-[2-(trifluoromethyl)benzoyl]-thieno[2,3-d]pyrimidine-2,4(1H,3H)-dione,

6-[Hydroxy[2-(trifluoromethyl)phenyl]methyl]-5-(1H-imidazol-2-ylthio)-3-methyl-1-(2-methylpropyl)-thieno[2,3-d]pyrimidine-2,4(1H,3H)-dione,

6-[Hydroxy[2-(trifluoromethyl)phenyl]methyl]-3-methyl-1-(2-methylpropyl)-5-(1H-1,2,4-triazol-3-ylthio)-thieno[2,3-d]pyrimidine-2,4(1H,3H)-dione,

3-Methyl-1-(2-methylpropyl)-5-(2-thiazolylthio)-6-[[2-(trifluoromethyl)-phenyl]methyl]-thieno[2,3-d]pyrimidine-2,4(1H,3H)-dione,

3-Methyl-1-(2-methylpropyl)-5-[(5-methyl- 1H-1,2,4-triazol-3-yl)thio]-6-[[2-(trifluoromethyl)phenyl]methyl]-thieno[2,3-d]pyrimidine-2,4(1H,3H)-dione dihydrochioride,

5-(1H-Imidazol-2-ylthio)-3-methyl-1-(2-methylpropyl)-6-[[2-(trifluoromethyl)phenyl]methyl]-thieno[2,3-d]pyrimidine-2,4(1H,3H)-dione,

3-Methyl-1-(2-methylpropyl)-5-(1,3,4-thiadiazol-2-ylthio)-6-[2-(trifluoromethyl)benzyl]-thieno[2,3 -d]pyrimidine-2,4(1H,3H)-dione,

5-[(5-Amino-1,3,4-thiadiazol-2-yl)thio]-3-methyl-1-(2-methylpropyl)-6-[2-(trifluoromethyl)benzyl]-thieno[2,3 -d]pyrimidine-2,4(1H,3H)-dione,

3-Methyl-1-(2-methylpropyl)-5-(2-thienylthio)-6-[[2-(trifluoromethyl)phenyl]methyl]-thieno[2,3-d]pyrimidine-2,4(1H,3H)-dione,

3-Methyl-1-(2-methylpropyl)-5-[(5-methyl-1H-1,2,4-triazol-3-yl)sulfonyl]-6-[[2-(trifluoromethyl)phenyl]methyl]-thieno[2,3-d]pyrimidine-2,4(1H,3H)-dione,

5-(1H-Imidazol-2-ylsulfonyl)-3-methyl-1-(2-methylpropyl)-6-[[2-(trifluoromethyl)phenyl]methyl]-thieno[2,3-d]pyrimidine-2,4(1H,3H)-dione,

6-[Hydroxy[2-(trifluoromethyl)phenyl]methyl]-3-methyl-1-(2-methylpropyl)-5-(1H-1,2,4-triazol-3-ylsulfonyl)-thieno[2,3-d]pyrimidine-2,4(1H,3H)-dione,

3-Methyl-1-(2-methylpropyl)-5-(1,3-thiazol-2-ylsulfonyl)-6-[2-(trifluoromethyl)benzyl]-thieno[2,3-d]pyrimidine-2,4(1H,3H)-dione,

3-Methyl-1-(2-methylpropyl)-5-(1,3,4-thiadiazol-2-ylsulfonyl)-6-[2-(trifluoromethyl)benzyl]-thieno[2,3-d]pyrimidine-2,4(1H,3H)-dione,

5-[(3-Bromo-1H-1,2,4-triazol-5-yl)thio]-3-methyl-1-(2-methylpropyl)-6-[2-(trifluoromethyl)phenylmethyl]-thieno[2,3 -d]pyrimidine-2,4(1H,3H)-dione,

3-Methyl-1-(2-methylpropyl)-5-[(5-oxo-4,5-dihydro-1H-1,2,4-triazol-3-yl)thio]-6-[2-(trifluoromethyl)phenylmethyl]-thieno[2,3-d]pyrimidine-2,4(1H,3H)-dione,

3-Methyl-1-(1-methylethyl)-5-(1,3-thiazol-2-ylthio)-6-[2-(trifluoromethyl)benzyl]-thieno[2,3-d]pyrimidine-2,4(1H,3H)-dione,

3-Methyl-1-(1-methylethyl)-5-(1,3-thiazol-2-ylsulfonyl)-6-[2-(trifluoromethyl)benzyl]thieno[2,3-d]pyrimidine-2,4(1H,3H)-dione,

1-(Cyclopropylmethyl)-3-methyl-5-(2-thienylcarbonyl)-6-[2-(trifluoromethyl)phenylmethyl]-thieno[2,3-d]pyrimidine-2,4(1H,3H)-dione,

6-(9H-Fluoren-9-yl)-3-methyl-1-(2-methylpropyl)-5-(1H-1,2,4-triazol-5-ylsulfonyl)-thieno[2,3-d]pyrimidine-2,4(1H,3H)-dione,

6-(1H-Indol-3-ylcarbonyl)-3-methyl-1-(2-methylpropyl)-5-(1H-1,2,4-triazol-3-ylthio)-thieno[2,3-d]pyrimidine-2,4(1H,3H)-dione,

6-(Hydroxy-(1H-indol-3-yl)methyl)-3-methyl-1-(2-methylpropyl)-5-(1H-1,2,4-triazol-3-ylthio)-thieno[2,3-d]pyrimidine-2,4(1H,3H)-dione,

3-Methyl-1-(2-methylpropyl)-6-(4-quinolinylcarbonyl)-5-(1,3-thiazol-2-ylthio)-thieno[2,3-d]pyrimidine-2,4(1H,3H)-dione,

6-[Hydroxy-(4-quinolinyl)-methyl]-3-methyl-1-(2-methylpropyl)-5-(1,3-thiazol-2-ylthio)-thieno[2,3-d]pyrimidine-2,4(1H,3H)-dione,

6-(1-Benzothien-3-ylmethyl)-3-methyl-1-(2-methylpropyl)-5-(4H-1,2,4-triazol-3-ylthio)-thieno[2,3-d]pyrimidine-2,4(1H,3H)-dione,

6-(1H-Benzimidazol-1-ylmethyl)-3-methyl-1-(2-methylpropyl)-5-(2-thienylthio)-thieno[2,3-d]pyrimidine-2,4(1H,3H)-dione,

6-(1H-Indol-2-ylmethyl)-3-methyl-1-(2-methylpropyl)-5-(2-thienylthio)-thieno[2,3-d]pyrimidine-2,4(1H,3H)-dione,

6-[(2-Chloro-6-methyl-1H-benzimidazol-1-yl)methyl]-3-methyl-1-(2-methylpropyl)-5-(2-thienylthio)-thieno[2,3-d]pyrimidine-2,4(1H,3H)-dione,

6-[(1,3-Dihydro-1,3-dioxo-2H-isoindol-2-yl)methyl]-3-methyl-1-(2-methylpropyl)-5-(2-thienylthio)-thieno[2,3-d]pyrimidine-2,4(1H,3H)-dione,

6-[Hydroxy[6-(trifluoromethyl)-2-pyridinyl]methyl]-3-methyl-1-(2-methylpropyl)-5-(2-thienylthio)-thieno[2,3-d]pyrimidine-2,4(1H,3H)-dione

and their pharmaceutically acceptable salts and solvates.

The present invention further provides a process for the preparation of a compound of formula (I) as defined above which comprises,

(a) when R represents C(O)Ar¹ and X represents a sulphur atom, reacting a compound of general formula

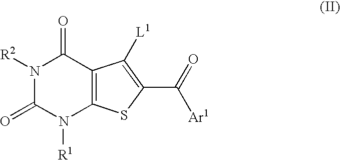

wherein L¹ represents a leaving group (e.g. a halogen atom such as bromine) and R¹, R² and Ar¹ are as defined in formula (I), with a compound of general formula (III), HS—Ar², wherein Ar² is as defined in formula (I), in the presence of a base (e.g. sodium hydride); or

(b) when R represents —C(R⁴)(R⁵)Ar¹, R⁴ represents a hydrogen atom, R⁵ represents hydroxyl and X represents a sulphur atom, reacting a corresponding compound of formula (I) in which R represents C(O)Ar¹ with a suitable reducing agent (e.g. sodium borohydride); or

(c) when R represents —C(R⁴)(R⁵)Ar¹, R⁴ represents a hydrogen atom, R⁵ represents a hydrogen atom and X represents a sulphur atom, reacting a corresponding compound of formula (I) in which R represents C(O)Ar¹ with a suitable reducing agent (e.g. BH₃.Me₂NH), in the presence of a Lewis acid (e.g. TiCl₄); or

(d) when R represents —C(R⁴)(R⁵)Ar¹ and R⁴ represents C₁₋₄ alkyl, reacting a corresponding compound of formula (I) in which R represents C(O)Ar¹ with a suitable Grignard reagent (e.g. C₁₋₄ alkylMgBr), followed by reaction with a dehydrating agent (e.g. methanesulphonyl chloride) in the presence of abase (e.g. triethylamine or 1,8-diazabicyclo[5.4.0]undec-7-ene, DBU), and then followed by a hydrogenation reaction; or

(e) when X represents SO or SO₂, reacting a corresponding compound of formula (I) in which X is a sulphur atom with a suitable oxidising agent; or

(f) when X represents CH(OH), reacting a compound of general formula

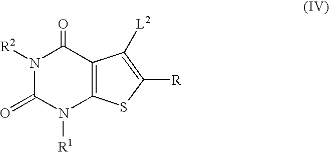

wherein L² represents a leaving group (e.g. a halogen atom such as bromine) and R, R¹ and R² are as defined in formula (I), with a suitable Grignard reagent (e.g. ethyl magnesium bromide) and then with a compound of general formula (V), Ar²CHO, wherein Ar² is as defined in formula (I); or

(g) when X represents C(O), reacting a corresponding compound of formula (I) in which X represents CH(OH) with a suitable oxidising agent (e.g. oxalyl chloride in dimethyl sulfoxide); or

(h) when X represents a sulphur atom, reacting a compound of formula (IV) as defined in (f) above with a suitable alkyl lithium compound or Grignard reagent and then with a compound of general formula (VI), Ar²—S—S—Ar², wherein Ar² is as defined in formula (I);

and optionally after (a), (b), (c), (d), (e), (f), (g) or (h) forming a pharmaceutically acceptable salt or solvate of the compound of formula (I) obtained.

Process (a) is conveniently carried out in an inert, aprotic solvent such as tetrahydrofuran at a temperature in the range from −20° C. to 100° C., preferably from 10° C. to 30° C.

Process (b) is conveniently carried out in an inert solvent such as ethanol at a temperature in the range from −20° C. to 100° C., preferably from 10° C. to 30° C.

Process (c) may be carried out in an inert, aprotic solvent such as dichlorometlhalne at a temperature in the range from −20° C. to 100° C., preferably from −10° C. to 30° C.

Process (d), first step is performed in an inert solvent such as tetrahydrofuran at a temperature in the range from −50° C. to 30° C., preferably from −20° C. to 20° C. The resulting adduct is preferably contacted in the second step with the dehydrating agent and the base in an inert solvent such as dichloromethane at a temperature in the range from −50° C. to 30° C., preferably from −10° C. to 20° C. In the third step, the hydrogenation reaction is conveniently carried out using a palladium on carbon catalyst in ethanol at a hydrogen pressure of 1 to 5×10⁵ Nm⁻² and at a temperature in the range from 10° C. to 30° C.

Process (e) is conveniently carried out in an inert, aprotic solvent such as dichloromethane at a temperature in the range from −20° C. to 100° C., preferably from 0° C. to 30° C. Suitable oxidising agents that may be used include 3-chloroperoxybenzoic acid or potassium peroxymonosulphate, commercially sold under the trade mark “OXONE”.

In process (f), reaction with the Grignard reagent may be performed in an inert, aprotic solvent such as tetrahydrofuran at a temperature in the range from −20° C. to 50° C., preferably from 0° C. to 25° C. Subsequent reaction with the compound of general formula (VI) is conveniently carried out in the same solvent at a temperature in the range from −20° C. to 100° C., preferably from 0° C. to 25° C.

Process (g) is conveniently carried out using oxalyl chloride in a dimethyl sulphoxide solution at a temperature in the range from −78° C. to −20° C., preferably from −78° C. to −50° C.

Both steps of process (h) are conveniently performed in an inert, aprotic solvent such as tetrahydrofuran at a temperature in the range from −78° C. to 50° C.

Compounds of formula (II) in which L¹ represents, for example, a bromine atom may be prepared by reacting a compound of general formula

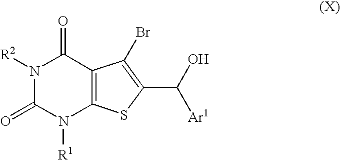

wherein R¹, R² and Ar¹ are as defined in formula (I), with an oxidising agent such as oxalyl chloride in dimethyl sulfoxide at a temperature in the range from −30° C. to 30° C.

Compounds of formula (X) may be prepared by reacting a compound of general formula

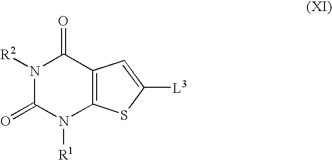

wherein L³ represents a halogen atom (e.g. bromine) and R¹ and R² are as defined in formula (I), with a lithium dialkylamide such as lithium diisopropylamide in an inert solvent (e.g. tetrahydrofuran) and at a temperature in the range from −78° C. to 0° C., followed by reaction with a compound of general formula (XII), Ar¹CHO, at a temperature in the range from −78° C. to 0° C.

Compounds of formula (XI) in which L³ represents, for example, a bromine atom may be prepared by reacting a compound of general formula

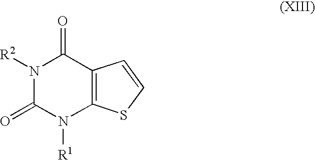

wherein R¹ and R² are as defined in formula (I), with bromine in an inert solvent (e.g. dichloromethane) at a temperature in the range from −20° C. to 50° C.

Compounds of formula (IV) in which R represents —C(O)Ar¹ are equivalent to compounds of formula (II) and may be prepared as described above.

Compounds of formula (IV) in which R represents —C(R⁴)(R⁵)Ar¹, R⁴ represents a hydrogen atom and R⁵ represents hydroxyl are equivalent to compounds of formula (X) and may be prepared as described above.

Compounds of formula (IV) in which R represents —C(R⁴)(R⁵)Ar¹, R⁴ represents a hydrogen atom and R⁵ represents a hydrogen atom may be prepared by reacting a compound of formula (X) with trifluoroacetic acid/triethylsilane at a temperature in the range from −20° C. to 50° C., optionally in the presence of an inert solvent such as dichloromethane.

Compounds of formula (IV) in which R represents —C(R⁴)(R⁵)Ar¹ and R⁴ represents C₁₋₄ alkyl, may be prepared by reacting a compound of formula (II) by a process analogous to process (d) above.

Compounds of formula (IV) in which R represents Ar³ may be prepared by reacting a compound of formula (XI) with a lithium dialkylamide such as lithium diisopropylamide in an inert solvent (e.g. tetrahydrofuran) at a temperature in the range from −78° C. to 0° C., followed by reaction with acenaphlithenone, indanone or fluorenone at a temperature in the range from −78° C. to 0° C. in the same solvent, and then reduction of the adduct using triethylsilane in the presence of a strong acid such as trifluoroacetic acid either in the absence of solvent or in an inert solvent (e.g. dichloromethane) at a temperature in the range from 0° C. to 30° C.

Compounds of formulae (III), (V), (VI), (XII) and (XIII) are either commercially available, are well known in the literature or may be prepared easily using known techniques.

It will be appreciated by those skilled in the art that in the processes of the present invention certain functional groups such as hydroxyl or amino groups in the starting reagents or intermediate compounds may need to be protected by protecting groups. Thus, the preparation of the compounds of formula (I) may involve, at an appropriate stage, the removal of one or more protecting groups.

The protection and deprotection of functional groups is fully described in ‘Protective Groups in Organic Chemistry’, edited by J. W. F. McOmie, Plenum Press (1973), and ‘Protective Groups in Organic Synthesis’, 2nd edition, T. W. Greene & P. G. M. Wuts, Wiley-Interscience (1991).

The compounds of formula (I) above may be converted to a pharmaceutically acceptable salt or solvate thereof, preferably an acid addition salt such as a hydrochloride, hydrobromide, phosphate, acetate, fumarate, maleate, tartrate, citrate, oxalate, methanesulfonate or p-toluenesulfonate, or an alkali metal salt such as a sodium or potassium salt.

Certain compounds of formula (I) are capable of existing in stereoisomeric forms. It will be understood that the invention encompasses all geometric and optical isomers, including atropisonlers, of the compounds of formula (I) and mixtures thereof including racemates. Tautomers and mixtures thereof also form an aspect of the present invention.

Isomers may be resolved or separated by conventional techniques, e.g. chromatography or fractional crystallisation. Enantiomers may be isolated by separation of a racemic or other mixture of the compounds using conventional techniques (e.g. chiral HPLC). Alternatively the desired optical isomers may be made by reaction of the appropriate optically active starting materials under conditions which will not cause racenusation, or by derivatisation, for example with a homochiral acid followed by separation of the diastereomeric derivatives by conventional means (e.g. HPLC, chromatography over silica) or may be made with achiral starting materials and chiral reagents. All stereoisomers are included within the scope of the invention.

The compounds of the invention may be isolated from their reaction mixtures using conventional techniques.

The compounds of the invention are useful because they possess pharmacological activity in human and non-human animals. They are therefore indicated as pharmaceuticals for use in the (prophylactic) treatment of autoimmune, inflammatory, proliferative and hyperproliferative diseases and immunologically-mediated diseases including rejection of transplanted organs or tissues and Acquired Immunodeficiency Syndrome (AIDS).

Examples of these conditions are:

(1) (the respiratory tract) obstructive airways diseases including chronic obstructive pulmonary disease (COPD); asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia;

(2) (bone and joints) rheumatoid arthritis, seronegative spondyloarthropathies (including anklosing spondylitis, psoriatic arthritis and Reiter's disease), Behcet's disease, Sjogren's syndrome and systemic sclerosis;

(3) (skin) psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis;

(4) (gastrointestinal tract) Coeliac disease, proctitis, eosinopilic gastro-enteritis, mastocytosis, Crohn's disease, ulcerative colitis, food-related allergies which have effects remote from the gut, e.g., migraine, rhinitis and eczema;

(5) (other tissues and systemic disease) multiple sclerosis, atherosclerosis, Acquired Immunodeficiency Syndrome (AIDS), lupus erythematosus, systemic lupus, erythematosus, Hashimoto's thyroiditis, myasthenia gravis, type I diabetes, nephrotic syndrome, eosinophilia fascitis, hyper IgE syndrome, lepromatous leprosy, sezary syndrome and idiopathic thrombocytopenia pupura;

(6) (allograft rejection) acute and chronic following, for example, transplantation of kidney, heart, liver, lung, bone marrow, skin and cornea; and chronic graft versus host disease; and

(7) cancer.

Accordingly, the present invention provides a compound of fonnula (I) or a pharmaceutically acceptable salt or solvate thereof as hereinbefore defined for use in therapy.

In another aspect, the invention provides the use of a compound of formula (I) or a pharmaceutically acceptable salt or solvate thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention further provides a method of effecting immunosuppression (e.g. in the treatment of allograft rejection) which comprises administering to a patient a therapeutically effective amount of a compound of formula (I) or a pharmaceutically acceptable salt or solvate thereof as hereinbefore defined.

The invention still furter provides a method of treating, or reducing the risk of, an obstructive airways disease (e.g. asthma or COPD) in a patient suffering from, or at risk of, said disease, which comprises administering to the patient a therapeutically effective amount of a compound of formula (I) or a pharmaceutically acceptable salt or solvate thereof as hereinbefore defined.

For the above-mentioned therapeutic uses the dosage administered will, of course, vary with the compound employed, the mode of administration, the treatment desired and the disorder indicated. However, in general, for effecting immunosuppression, the daily dosage of the compound of formula (I) will be in the range fiom 0.1 mg/kg, preferably from 0.3 mg/kg, more preferably from 0.5 mg/kg and still more preferably from 1 mg/kg up to and including 30 mg/kg. For the treatment of obstructive airways diseases, the daily dosage of the compound of formula (I) will typically be in the range from 0.001 mg/kg to 30 mg/kg.

The compounds of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the phannaceutical composition will preferably comprise from 0.05 to 99% w (per cent by weight), more preferably less than 80% w, e.g. from 0.10 to 70% w, and even more preferably less than 50% w, of active ingredient, all percentages by weight being based on total composition.

Thus, the present invention also provides a pharmaceutical composition comprising a compound of formula (I) or a pharmaceutically acceptable salt or solvate thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt or solvate thereof as hereinbefore defined, with a pharmaceutically acceptable adjuvant, diluent or carrier.

The pharmaceutical composition of the invention may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally.

The invention will now be further explained by reference to the following illustrative examples.

### EXAMPLE 1

5-[3-Furyl(hydroxy)methyl]-1-isobutyl-3-methyl-6-[2-(trifluoromethyl)benzyl]thieno[2,3-d]pyrimidine-2,4(1H,3H)-dione

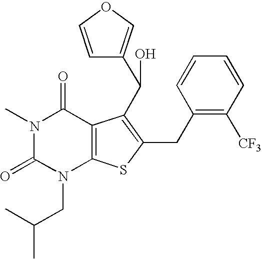

a) 5-Bromo-6-{hydroxy[2-(trifluoromethyl)phenyl]methyl]-1-isobutyl-3-methyl-thieno[2,3-d]pyrimidine-2,4(1H,3H)-dione

1-Isobutyl-3-methyl-thieno[2,3-d]pyrimidine-2,4(1H,3H)-dione (20 g) was dissolved in DCM (100 mls) and cooled to 0° C. before bromine (4.94 mls) was added dropwise. Ten minutes after full addition the mixture was concentrated in vacuo, redissolved in ethyl acetate and washed with a 50% aqueous sodium hydrogen bicarbonate followed by sodium thiosulfate and then brine. The ethyl acetate was separated, dried over magnesium sulfate, filtered and concentrated in vacuo to a pale yellow solid. This solid was stored under a high vacuum for 1 hour before being dissolved in tetrahydrofuran (THF) (160 mls) and cooled to −78° C. To this solution was slowly added lithium diisopropylamide (LDA) (126 mls of 1M solution in isohexanes/THF). After complete addition the reaction was stirred for 30 minutes before 2-trifluoromethyl benzaldehyde (16.61 mls) was added neat in 1 aliquot. The resultant solution was allowed to stir 2 hrs before 50 mls of water was added and the cooling bath removed. Once the reaction mixture had reached room temperature it was concentrated in vacuo and partitioned between ethyl acetate and water. The ethyl acetate was collected, dried over magnesium sulfate, filtered and concentrated in vacuo to a dark oil. The oil was purified via silica chromatography, eluting with 4:1 isohexane:ethyl acetate to yield 33.47 g (81%) of the subtitle compound.

MS: [M−OH]⁺ 474

b) 5-Bromo-1-isobutyl-3-methyl-6-[2-(trifluoromethyl)benzyl]-thieno[2,3-d]pyrimidine-2,4(1H,3H)-dione

5-Bromo-6-{hydroxy[2-(trifluoromethyl)phenyl]methyl}-1-isobutyl-3-methyl-thieno[2,3-d]pyrimidine-2,4(1H,3H)-dione (33.47 g) was dissolved in triethyl silane (100 mls) and cooled to 0° C. before trifluoroacetic acid (100 mls) was added dropwise. After complete addition the resultant thick suspension was diluted by the addition of dichloromethane (DCM) (100 mls) and the reaction was stirred at room temperature for 18 hours. The reaction mixture was then concentrated in vacuo and partitioned between DCM and 50% aqueous sodium hydrogen carbonate. The organic layer was collected, dried over magnesium sulfate, filtered and concentrated in vacuo. The resultant off white solid was stirred vigorously with isohexane for 1 hour before being collected by filtration to yield 27.286 g (84.5%) of the subtitle compound.

MS: [M+H]⁺ 476

c) 5-[3-Furyl(hydroxy)methyl]-1-isobutyl-3-methyl-6-[2-(trifluoromethyl)benzyl]-thieno[2,3-d]pyrimidine-2,4(1H,3H)-dione

5-Bromo-1-isobutyl-3-methyl-6-[2-(trifluoromethyl)benzyl]-thieno[2,3-d]pyrimidine-2,4(1H,3H)-dione (75 mg) was dissolved in THF (5 mls) under nitrogen to which ethyl magnesium bromide (1M, 240 μl) was added in 1 aliquot. After 15 minutes furan-3-carboxaldehyde (20 μl) was added and the reaction left for 2 hours before being concentrated in vacuo and loaded directly on an Isolute silica cartridge and eluting off with 2:1 isohexane:diethyl ether to yield 12 mg of the title compound.

MS: [M−OH]⁺ 475

¹H NMR (d₆-DMSO) δ0.85 (6H, d), 1.99-2.13 (1H, dm), 3.26 (3H, s), 3.65 (2H, d), 4.39-4.53 (2H, dd), 6.24 (1H, d), 6.36 (1H, s), 6.46 (1H, d), 7.31(1H, d), 7.39 (1H, s), 7.47 (1H, t), 7.54 (1H, s), 7.59 (1H, t), 7.73 (1H, d).

### EXAMPLE 2

5-[2-Furyl(hydroxy)methyl]-1-isobutyl-3-methyl-6-[2-(trifluoromethyl)benzyl]-thieno[2,3-d]pyrimidine-2,4(1H,3H)-dione

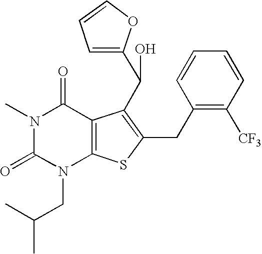

The title compound was prepared by the method of Example 1c), using furan-2-carboxaldehyde.

MS: [M−OH]⁺ 475

¹H NMR (d₆-DMSO) δ0.87 (6H, d), 2.08 (1H, m), 3.24 (3H, s), 3.65 (2H, d), 4.30-4.6 (2H, dd), 6.17 (1H, d), 6.34 (1H, d), 6.43 (1H, d), 6.63 (1H, d), 7.32 (1H, d), 7.47 (1H, t), 7.55 (1H, s), 7.59 (1H, t), 7.73 (1H, d).

### EXAMPLE 3

5-[Hydroxy(5-methyl-2-thienyl)methyl]-1-isobutyl-3-methyl-6-[2-(trifluoromethyl)benzyl]-thieno[2,3-d]pyrimidine-2,4(1H,3H)-dione

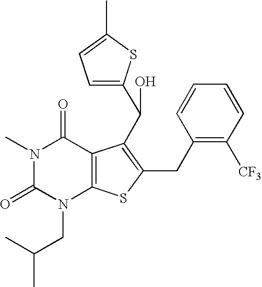

The title compound was prepared by the method of Example 1c), using 5-methyl-2-thiophenecarbaldehyde.

MS: [M−OH]⁺ 505

¹H NMR (d₆-DMSO) δ0.88 (6H, d), 2.07-2.14 (1H, m), 2.17 (3H, s), 3.24 (3H, s), 3.65 (2H, d), 4.33-4.71(2H, dd), 6.37 (1H, d), 6.80 (1H, d), 6.90 (1H, d), 7.22 (1H, d), 7.31 (1H, d), 7.46 (1H, t), 7.58 (1H, t), 7.74 (1H, d)

### EXAMPLE 4

5-[Hydroxy(3-pyridinyl)methyl]-1-isobutyl-3-methyl-6-[2-(trifluoromethyl)benzyl]-thieno[2,3-d]pyrimidine-2,4(1H,3R)-dione

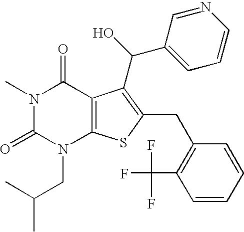

The title compound was prepared by the method of Example 1c), using nicotiinaldehyde.

MS: [M+H]⁺ 504

¹H NMR (d₆-DMSO) δ0.85 (6H, d), 2.07-2.13 (1H, m), 3.26 (3H, s), 3.63-3.67 (2H, d), 4.36-4.53 (2H, dd), 6.42 (1H, d), 6.78 (1H, d), 7.18 (1H, d), 7.28 (1H, m), 7.44 (1H, t), 7.53 (1H, t), 7.71 (1H, d), 8.39 (1H, d), 8.52 (1H, d)

### EXAMPLE 5

5-[Hydroxy(2-thienyl)methyl]-1-isobutyl-3-methyl-6-[2-(trifluoromethyl)benzyl]thieno[2,3-d]pyrimidine-2,4(1H,3H)-dione

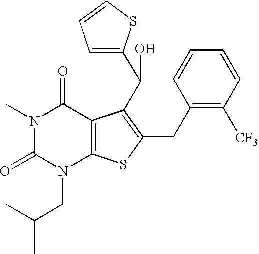

The title compound was prepared by the method of Example 1c), using 2-thiophenecarbaldehyde.

MS: [M−OH]⁺ 491

¹H NMR (d₆-DMSO) δ0.86 (6H, d), 2.07-2.14 (1H, m), 3.24 (3H, s), 3.67 (2H, d), 4.37-4.63 (2H, dd), 6.67 (1H, d), 6.81 (1H, d), 6.91 (1H, d), 7.29 (1H, d), 7.37 (1H, d), 7.47 (1H, t), 7.58 (1H, t), 7.73 (1H, d)

### EXAMPLE 6

5-[Hydroxy(3-thienyl)methyl]-1-isobutyl-3-methyl-6-[2-(trifluoromethyl)benzyl]-thieno[2,3-d]pyrimidine-2,4(1H,3H)-dione

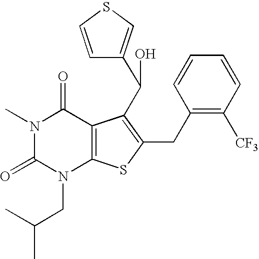

The title compound was prepared by the method of Example 1c, using 3-thiophenecarbaldehyde.

MS: [M−OH]⁺ 491

¹H NMR (d₆-DMSO) δ0.86 (6H, d), 2.07-2.17 (1H, m), 3.26(3H, s), 3.62-3.68 (2H, m), 4.38-4.50 (2H, dd), 6.35 (1H, d), 6.65 (1H, d), 6.91(1H, d), 6.98 (1H, d), 7.16 (1H, d), 7.23 (1H, t), 7.43 (1H, t), 7.53 (1H, d), 7.71 (1H, d)

### EXAMPLE 7

1-isobutyl-3-methyl-5-[(1-methyl-1H-pyrrol-2-yl)carbonyl]-6-[2-(trifluoromethyl)benzyl]-thieno[2,3-d]pyrimidine-2,4(1H,3H)-dione

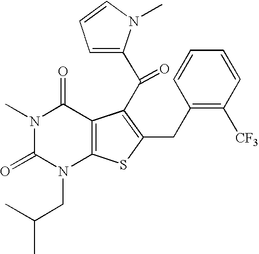

5-Bromo-1-isobutyl-3-methyl-6-[2-(trifluoromethyl)benzyl]-thieno[2,3-dupyrimidine-2,4(1H,3H)-dione (100 mg) was dissolved in THF(1 ml) under nitrogen to which ethyl magnesium bromide (1M, 210 μl) was added in 1 aliquot. After 15 minutes 1-methyl-1H-pyrrole-2-carbaldehyde (1M in THF, 210 μl) was added and the reaction was left for 1 hour. Water (2 ml) was added and the reaction was stored under high vacuum. Once all of the THF had evaporated, the water was extracted twice with DCM. This DCM solution was then added to a preformed mixture of oxalyl chloride(50 μl) and dimethyl sulphoxide (DMSO) (100 μl ) in DCM (1 ml) at −78° C. After 10 minutes triethylamine (260 μl) was added and the reaction was stirred for 30 minutes before being allowed to wami to room temperature. Once at room temperature the reaction was washed with water (5 ml) and evaporated to dryness. The residue was purified by silica chromatography, eluting with a 0 to 75% gradient of isohexane in diethyl ether to yield the title compound.

MS: [M+H]⁺ 504

¹H NMR (d₆-DMSO) δ0.87 (6H, d), 2.09-2.13 (1H, m), 3.12 (3H, s), 3.21 (3H,s), 3.66 (2H, d), 4.23 (2H, d), 6.31 (1H, d), 6.53 (1H, s), 7.22 (1H, d), 7.40 (1H, d), 7.47 (1H, t), 7.65 (1H, t), 7.72 (1H, d)

### EXAMPLE 8

1-Isobutyl-3-methyl-5-(3-thienylcarbonyl)-6-[2-(trifluoromethyl)benzyl]-thieno[2,3-d]pyrimidine-2,4(1H,3H)-dione

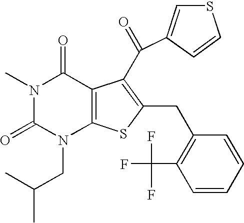

The title compound was prepared by the method of Example 7, using 3-thiophenecarbaldehyde.

MS: [M+H]⁺ 507

¹H NMR (d₆-DMSO) δ0.88 (6H, d), 2.13-2.19 (1H, m), 3.14 (3H, s) 3.66 (2H, d), 4.15 (2H, d), 7.44-7.49 (3H, m), 7.60-7.64 (2H, m), 7.70 (1H, d), 8.17 (1H, s).

### EXAMPLE 9

1-Isobutyl-3-methyl-5-[(3-methyl-2-pyridinyl)carbonyl]-6-[2-(trifluoromethyl)benzyl]-thieno[2,3-d]pyrimidine-2,4(1H,3H)-dione

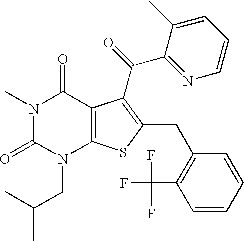

The title compound was prepared by the method of Example 7, using 3-methyl-2-pyridinecarbaldehyde

MS: [M+H]⁺ 516

¹H NMR (d₆-DMSO) δ0.90 (6H, d), 2.15-2.19 (1H, m), 2.41 (3H, s), 3.32 (3H, s), 3.71 (2H, d), 4.19 (2H, d), 7.45-7.47 (2H, m), 7.55 (1H, d), 7.59 (1H, t), 7.68 (1H, d), 7.83 (1H, d), 7.89 (1H, t).

### EXAMPLE 10

1-Isobutyl-3-methyl-5-(3-pyridinylcarbonyl)-6-[2-(trifluoromethyl)benzyl]-thieno[2,3-d]pyrimidine-2,4(1H,3H)-dione

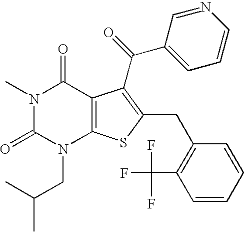

The title compound was prepared by the method of Example 7, using 3-pyridinecarbaldehyde

MS: [M+H]⁺ 502

¹H NMR (d₆-DMSO) δ0.92 (6H, d), 2.13-2.19 (1H, m), 3.09 (3H, s) 3.71 (2H, d), 4.22 (2H, d), 7.41 (1H, d), 7.45 (1H, t), 7.5 (lH, t), 7.57 (2H, m), 8.06 (1H, d), 8.75 (1H,d), 8.83 (1H, s).

### EXAMPLE 11

1-Isobutyl-3-methyl-5-(2-thienylcarbonyl)-6-[2-(trifluoromethyl)benzyl]-thieno[2,3-d]pyrimidine-2,4(1H,3H)-dione

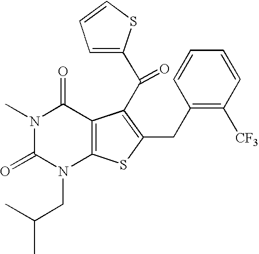

The title compound was prepared by the method of Example 7, using 2-thiophenecarbaldehyde

MS: [M+H]⁺ 507

¹H NMR (d₆-DMSO), δ0.90 (6H, d), 2.13-2.19 (1H, m), 3.14 (3H, s) 3.75 (2H, d), 4.19 (2H, d), 7.18 (1H, m), 7.41(1H, d), 7.42 (1H,t), 7.47 (1H, m), 7.61 (1H, t), 7.69 (1H, d), 8.08 (1H, d)

### EXAMPLE 12

3-Methyl-1-(2-methylpropyl)-5-(1H-1,2,4-triazol-3-ylthio)-6-[2-(trifluoromethyl)benzoyl]-thieno[2,3-d]pyrimidine-2,4(1H,3H)-dione

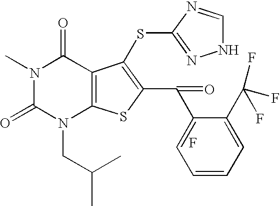

a) 5-Bromo-3-methyl-1-(2-methylpropyl)-6-[2-(trifluoromethyl)benzoyl]-thieno[2,3-d]pyrimidine-2,4(1H,3H)-dione

A solution of 5-bromo-6-[hydroxy[2-(trifluoromethyl)phenyl]methyl]-3-methyl-1-(2-methylpropyl)-thieno[2,3-d]pyrimidine-2,4(1H,3H)-dione, (6.44 g) (prepared as described in Example 1 part a)), 4-methyl morpholine-N-oxide (2.3 g), powdered 4 Å sieves (6.55 g) and tetra-n-propylammonium perruthenate (0.23 g) in dry dichloromethane (120 ml) was stirred for 1 hour at room temperature. The suspension was filtered through Celite and the filtrate was concentrated under reduced pressure. The residue was purified by column chromatography, eluting with dichloromethane, to give the sub-title compound as a pale cream foam (6.08 g).

MS (APCI) 490.9{M+H]+

b) 3-Methyl-1-(2-methylpropyl)-5-(1H-1,2,4-triazol-3-ylthio)-6-[2-(trifluoromethyl)benzoyl]-thieno[2,3-d]pyrimidine-2,4(1H,3H)-dione

3-Mercapto-1,2,4-triazole (0.45 g) in dry tetrahydrofuran (10 ml) was added dropwise to a suspension of sodium hydride (0.196 g, 60% oil dispersion) in dry tetrahydrofuran (30 ml) at room temperature under nitrogen. After 15 minutes, a solution of 5-bromo-3-methyl-1-(2-methylpropyl)-6-[2-(trifluoromethyl)benzoyl]-thieno[2,3-dpyrirdine-2,4(1H,3H-dione (2.0 g) in dry tetrahydrofiran (20 ml) was added dropwise and the reaction was stirred for 24 hours at room temperature. The solution was poured into water and extracted with ethyl acetate. The combined extracts were dried over magnesium sulfate, filtered and concentrated under reduced pressure. The residue was purified by flash silica chromatography eluting with 4% ethanol in dichloromethane to give the title compound as a yellow foam (2.0 g).

MS (APCI) 509.9[M+H]+

¹H NMR(CDCl₃) δ1.03 (6H,d), 2.34 (1H,septet), 3.30 (3H,s), 3.86 (2H,d), 7.49-7.60 (3H,m), 7.75 (1H,d), 7.98 (1H,s).

### EXAMPLE 13

3-Methyl-1-(2-methylpropyl)-5-(1H-1,2,4-triazol-3-ylthio)-6-[[2-(trifluoromethyl)phenyl]methyl]-thieno[2,3-d]pyrimidine-2,4(1H,3H)-dione

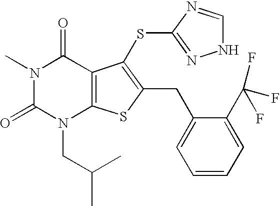

Titanium tetrachioride in dichloromethane (1.0M, 13.74 ml) was added to a solution of 3-methyl-1-(2-methylpropyl)-5-(1H-1,2,4-triazol-3-ylthio)-6-[2-(trifluoromethyl)benzoyl]-thieno[2,3-d]pyrimidine-2,4(1H,3H)-dione (2.0 g, as prepared in Example 12) in dry dichioromethane (80 ml) at 0° C. Dimethylamine-borane complex in dry dichoromethane (20 ml) was added dropwise. The solution was allowed to warm to room temperature and stirred for 3 hours. Hydrochloric acid (2.0M, 10 ml) was added to the reaction mixture and stirring continued for 5 minutes. The solution was extracted with dicliloromethane and the combined extracts were washed with brine, dried over anhydrous magnesium sulfate, filtered and concentrated under reduced pressure. The residue was purified by flash silica chromatography eluting with a gradient of 1-6% ethanol in dichloromethane to give a solid which was recrystallised from acetonitrile to give the title compound as a white solid (0.69 g).

m.p. 186-187° C.

MS (APCI) 496[M+H]+

¹H NMR (CDCl₃) δ0.92 (6H,d), 2.19 (1H,septet), 3.45 (3H,s), 3.70 (2H,d), 4.69 (2H,s), 7.30 (1H,d), 7.37 (1H,t), 7.47 (1H,t), 7.69 (1H,d), 8.02 (1H,s).

### EXAMPLE 14

3-Methyl-1-(2-methylpropyl)-5-(1H-1,2,4-triazol-3-ylsulfonyl)-6-[[2-(trifluoromethyl)phenyl]methyl]-thieno[2,3-d]pyrimidine-2,4(1H,3H)-dione

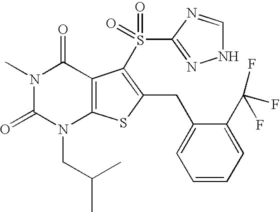

3-Chloroperoxybenzoic acid (0.22 g, 57-86%) was added to a suspension of 3-methyl-1-(2-methylpropyyl)-5-(1H- 1,2,4-triazol-3-ylthio)-6-[[2-(trifluoromethyl)phenyl]methyl]-thieno[2,3-d]pyrimidine-2,4(1H,3H)-dione (0.16 g, prepared as in Example 13) in dry dichloromethane (50 ml) and stirred at room temperature for 2 hours. The reaction mixture was diluted with ethyl acetate and washed with saturated sodium metabisulfite solution, saturated sodium bicarbonate solution, then brine. The organic layer was dried over anhydrous magnesium sulfate, filtered and concentrated under reduced pressure. The residue was purified by flash silica chromatography eluting with a gradient of 0-5% ethanol in dichloromethane to give the title compound as a white foam (0.092 g).

MS (ES+) 528{M+H]+

¹H NMR (CDCl₃) δ0.91 (6H,d), 2.16 (1H,septet), 3.26 (3H,s), 3.70 (2H,d), 5.03 (2H,s), 7.26-7.27 (1H,m), 7.44 (1H,t), 7.52-7.58 (2H,m), 7.73 (1H,d), 8.41 (1H,br.s).

By the method of Example 12), step b the following compounds were prepared:

### EXAMPLE 15

3-Methyl-1-(2-methylpropyl)-5-(2-thiazolylthio)-6-[2-(trifuoromethyl)benzoyl]-thieno[2,3-d]pyrimidine-2,4(1H,3H)-dione

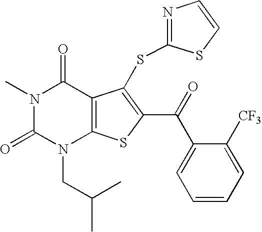

Prepared using 2-mercaptothiazole and purified by flash silica chromatography eluting with a gradient of 0-60% ethyl acetate in isohexane.

MS (APCI) 526[M+H]+

¹H NMR (CDCl₃) δ1.04 (6H,d), 2.37 (1H, septet), 3.29 (3H,s), 3.87 (2H,d), 7.12 (1H,d), 7.40-7.44 (2H,m), 7.52-7.60 (2H,m), 7.74 (1H,d).

### EXAMPLE 16

3-Methyl-1-(2-methylpropyl)-5-[[5-(methylthio)-1,3,4-thiadiazol-2-yl]thiol-6-[2-(trifluoromethyl)benzoyl]-thieno[2,3-d]pyrimidine-2,4(1H,3H)-dione

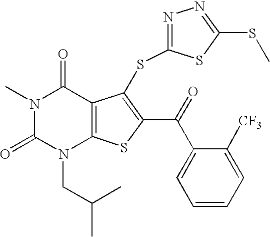

Prepared using 5-methylthio-1,3,4-thiadiazole-2-thiol and purified by flash silica chromatography eluting with a gradient of 0-60% ethyl acetate in isohexane.

MS (APCI) 573[M+H]+

¹H NMR (CDCl₃) δ1.04 (6H,d), 2.36 (1H, septet), 2.72 (3H,s), 3.30 (3H,s), 3.86 (2H,d), 7.46-7.47 (2H,m), 7.55-7.60 (1H,m), 7.77 (1H,br.d).

### EXAMPLE 17

3-Methyl-5-[(4-methyl-2-oxazolyl)thio]-1-(2-methylpropyl)-6-[2-(trifluoromethyl)benzoyl]-thieno[2,3-d]pyrimidine-2,4(1H,3H)-dione

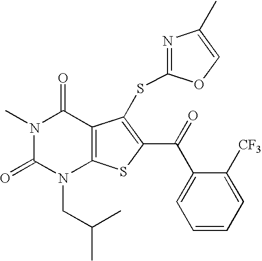

Prepared using 4-methyl-2-oxazolethiol and purified by flash silica chromatography eluting with 0-60% ethyl acetate in isohexane.

MS (APCI) 524[M+H]+

¹H NMR (CDCl₃) δ1.0 (6H,d), 2.07 (3H,s), 2.35 (1H,septet), 3.30 (3H,s), 3.86 (2H,d), 7.26-7.27 (1H,m), 7.43 (1H,d), 7.51 (1H,t), 7.59 (1H,t), 7.75 (1H,d).

### EXAMPLE 18

3-Methyl-1-(2-methylpropyl)-5-[(5-methyl-1H-1,2,4-triazol-3-yl)thiol-6-[2-(trifluoromethyl)benzoyl]-thieno[2,3-d]pyrimidine-2,4(1H,3H)-dione

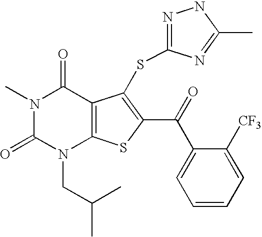

Prepared using 5-methyl-1H-1,2,4-triazole-3-thiol and purified by flash silica chromatography eluting with 10% ethanol in ethyl acetate.

m.p. 220-222° C.

MS (APCI) 524{M+H]+

¹H NMR(CDCl₃) δ0.98 (6H,d), 2.35 (1H,septet), 2.36 (3H,s), 3.30 (3H,s), 3.85 (2H,d), 7.50-7.60 (3H,m), 7.73 (1H,d).

### EXAMPLE 19

3-Methyl-1-(2-methylpropyl)-5-[(5-hydroxy-4-(1-methylethyl)-1H-1,2,4-triazol-3-yl)thio]-6-[2-(trifluoromethyl)benzoyl]-thieno[2,3-d]pyrimidine-2,4(1H,3H)-dione

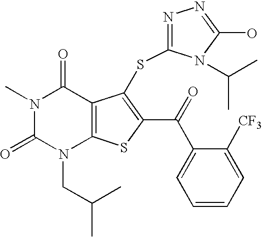

Prepared using 5-hydroxy-4-isopropyl-4H-1,2,4-triazole-3-thiol and purified by flash silica chromatography eluting with a gradient of 0-2% ethanol in dichloromethane.

MS (APCI) 568{M+H]+

¹H NMR(CDCl₃) δ0.86 (6H,d), 1.37 (6H,d), 2.34 (1H,septet), 3.31 (3H,s), 3.85 (2H,d), 4.17 (1H,quintet), 7.47-7.62 (3H,m), 7.76 (1H,d), 9.07 (1H,br.s).

### EXAMPLE 20

3-Methyl-1-(2-methylpropyl)-5-[(1-methyl-1H-tetrazol-5-yl)thio]-6-[2-(trifluoromethyl)benzoyl]-thieno[2,3-d]pyrimidine-2,4(1H,3H-dione

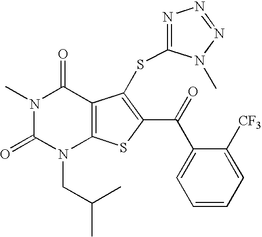

Prepared using 5-mercapto-1-methyltetrazole and purified by flash silica chromatography eluting with 2% ethanol in dichloroniethane.

MS (APCI) 525[M+H]+

¹H NMR (CDCl₃) δ1.02 (6H,d), 2.32 (1H,septet), 3.22 (3H,s), 3.82 (2H,d), 4.01 (3H,s), 7.50-7.62 (3H,m), 7.78 (1H,d).

### EXAMPLE 21

3-Methyl-1-(2-methylpropyl)-5[(5-methyl-1,3,4-thiadiazol-2-yl)thio]-6-[2-(trifluoromethyl)benzoyl]-thieno[2,3-d]pyrimidine-2,4(1H,3H)-dione

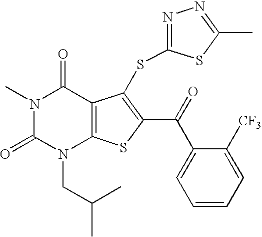

Prepared using 2-mercapto-5-methyl-1,3,4-thiadiazole and purified by flash silica chromatography eluting with a gradient of 20-100% ethyl acetate in isohexane.

MS (APCI) 541 [M+H]+

¹H NMR (CDCl₃) δ1.04 (6H,d), 2.36 (1H,septet), 2.69 (3H,s), 3.28 (3H,s), 3.87 (2H,d), 7.40-7.49 (2H,m), 7.53-7.60 (1H,rn), 7.76 (1H,d).

### EXAMPLE 22

5-(1H-Imidazol-2-ylthio)-3-methyl-1-(2-methylpropyl)-6-[2-(trifluoromethyl)bezoyl]-thieno[2,3-d]pyrimidine-2,4(1H,3H)-dione

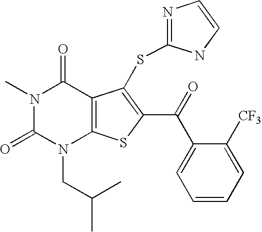

Prepared using 2-mercaptoimidazole and purified by flash silica chormnatography with a gradient for 0-6% ethanol in dichloromethane.

m.p. 123-127° C.

MS (APCI) 509.09[M+H]+

¹H NMR (CDCl₃) δ1.01 (6H,d), 2.32 (1H,septet), 3.39 (3H,s), 3.85 (2H,d), 6.99-7.03 (2H,m), 7.60-7.65 (1H,m), 7.70-7.71 (2H,m), 7.77 (1H,d).

### EXAMPLE 23

5-[[4-(1,1-Dimethylethyl)4H-1,2,4-triazol-3-yl]thio]-3-methyl-1-(2-methylpropyl)-6-[2-(trifluoromethyl)benzoyl]-thieno[2,3-d]pyrimidine-2,4(1H,3H)-dione

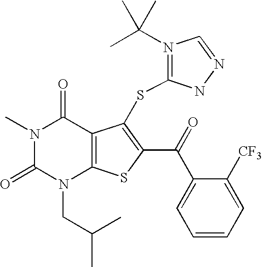

Prepared using 4-(1,1-dimethylethyl)-4H-1,2,4-triazole-3-thiol and purified by flash silica chromatography eluting with a gradient of 0-5% ethanol in dichlorornethane.

MS (APCI) 566[M+H]+

¹H NMR (CDCl₃) δ1.03 (6H,d), 1.55 (9H,d), 2.34 (1H,septet), 3.23 (3H,s), 3.85 (2H,d), 7.37 (1H,d), 7.47 (1H,t), 7.58 (1H,d), 7.73 (1H,d), 8.10 (1H,s).

### EXAMPLE 24

3-Methyl-1-(2-methylpropyl)-5-(2-thienylthio)-6-[2-(trifluoromethyl)benzoyl]-thieno[2,3-d]pyrimidine-2,4(1H,3H)-dione

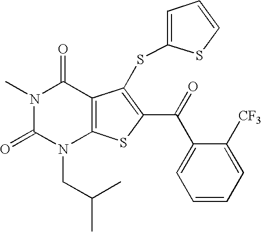

Prepared using 2-mercaptothiophene and purified by reversed phase mass-directed HPLC to give the title compound.

MS(APCI) 525[M+H]+

¹H NMR (CDCl₃) δ1.01 (6H,d), 2.33 (1H,septet), 3.35 (3H,s), 3.83 (2H,d), 6.85 (1H,t), 7.03 (1H,d), 7.24 (1H,d), 7.49-7.52 (1H,m), 7.60-7.63 (2H,m), 7.76-7.79 (1H,m).

### EXAMPLE 25

5-[(5-Amino-1,3,4-thiadiazol-2-yl)thio]-3-methyl-1-(2-methylpropyl)-6-[2-(trifluoromethyl)benzoyl]-thieno[2,3-d]pyrimidine-2,4(1H,3H)-dione

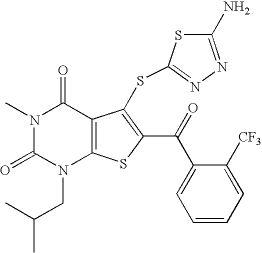

Prepared using 2-amino-5-mercapto-1,3,4-thiadiazole and purified by column chlromatography over silica, eluting with ethyl acetate then by re-crystallisation from ethyl acetate/isohexane.

MS (+ve APCI) 542 ((M+H)+)

¹H NMR (CDCl₃ ) δ1.03 (6H, d), 2.29-2.41 (1H, m), 3.32 (3H, s), 3.86 (2H, d), 4.97 (2H, s, br), 7.26-7.62 (3H, m), 7.76 (1H, d).

### EXAMPLE 26

3-Methyl-1-(2-methylpropyl)-5-(1,3,4-thiadiazol-2-ylthio)-6-[2-(trifluoromethyl)benzoyl]-thieno[2,3-d]pyrimidine-2,4(1H,3H)-dione

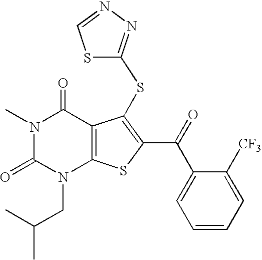

Prepared using 2-mercapto-1,3,4-thiadiazole and recrystallised from ethyl acetate/isohexane.

MS (+ve APCI) 527 ((M+H)+)

¹H NMR (DMSO-D6 ) δ0.98 (6H, d), 2.20-2.32 (1H, m), 3.12 (3H, s), 3.86 (2H, d), 0.76-7.63 (2H, m), 7.72-7.76 (2H, m), 7.89 (1H, d), 9.44 (1H, s).

### EXAMPLE 27

6-[Hydroxy[2-(trifluoromethyl)phenyl]methyl]-5-(1H-imidazol-2-ylthio)-3-methyl-1-(2-methylpropyl)-thieno[2,3-d]pyrimidine-2,4(1H,3,H)-dione

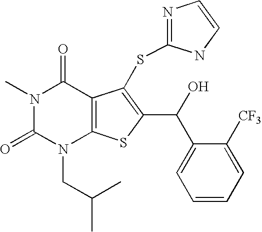

Sodium borohydride (0.008 g) was added to a solution of the product of example 15 viii) (0.10 g,) in dry ethanol and stirred at room temperature under nitrogen for 24 hours. The solution was concentrated under reduced pressure and the residue was partitioned between ethyl acetate and water. The organic layer was dried over anhydrous magnesium sulfate, filtered and concentrated under reduced pressure. The residue was purified by flash silica chromatography eluting with 10% ethanol in dichloromethane to give the title compound as a white foam (0.050 g).

MS (APCI) 511[M+H]+

¹H NMR (CDCl₃) δ0.86 (6H,dd), 2.11 (2H,m), 3.39 (3H,s), 3.53 (1H,dd), 3.71 (1H,dd), 7.05 (2H,s), 7.26 (1H,s), 7.50 (1H,t), 7.68-7.75 (2H,m), 8.23 (1H,d).

### EXAMPLE 28

6-[Hydroxy[2-(trifluoromethyl)phenyl]methyl]-3-methyl-1-(2-methylpropyl)-5-(1H-1,2,4-triazol-3-ylthio)-thieno[2,3-d]pyrimidine-2,4(1H,3H)-dione

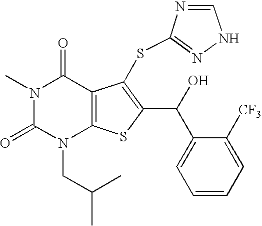

Prepared from the compound of Example 12 by the method of Example 27.

MS (APCI) 512 [M+H]+

¹H NMR (CDCl₃) δ0.89 (6H,dd), 2.14 (1H,septet), 3.39 (3H,s), 3.57 (1H,dd), 3.74 (1H,dd), 7.18 (1H,s), 7.52 (1H,t), 7.69-7.76 (2H,m), 8.11 (1H,s), 8.23 (1H,br.d).

By the method of Example 13 were prepared the following compounds:

### EXAMPLE 29

3-Methyl-1-(2-methylpropyl)-5-(2-thiazolylthio)-6-[[2-(trifluoromethyl)-phenyl]methyl]-thieno[2,3-d]pyrimidine-2,4(1H,3H)-dione

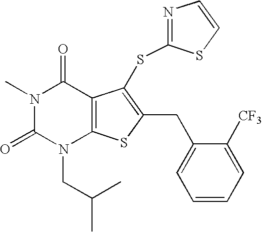

Prepared from the compound of Example 15.

m.p. 141-144° C.

MS (ES+) 512{M+H]+

¹H NMR (DMSO) δ0.87 (6H,dd), 2.11-2.16 (1H,m), 3.18 (3H,d), 3.71 (2H,d), 4.53 (2H,s), 7.40 (1H,d), 7.51 (1H,t), 7.54-7.57 (1H,m), 7.62-7.68 (2H,m), 7.77 (1H,d).

### EXAMPLE 30

3-Methyl-1-(2-methylpropyl)-5-[(5-methyl-1H-1,2,4-triazol-3yl)thiol-6-[[2-(trifluoromethyl)phenyl]methyl]-thieno[2,3-d]pyrimidine-2,4(1H,3H)-dione dihydrochloride

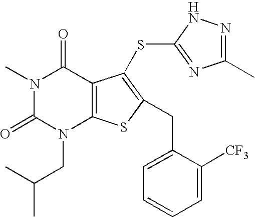

Prepared fom the compound of Example 18.

m.p. 154° C.

MS (APCI) 510[M+H]+

¹H NMR (DMSO) δ0.86 (6H,d), 2.12 (1H,septet), 2.25 (3H,s), 3.18 (3H,s), 3.53 (1H,br.s), 3.68 (2H,d), 4.42 (2H,s), 7.36 (1H,d), 7.48 (1H,t), 7.59 (1H,t), 7.75 (1H,d).

### EXAMPLE 31

5-(1H-Imidazol-2-ylthio)-3-methyl-1-(2-methylpropyl)-6-[[2-(trifluoromethyl)phenyl]methyl]-thieno[2,3-d]pyrimidine-2,4(1H,3H)-dione

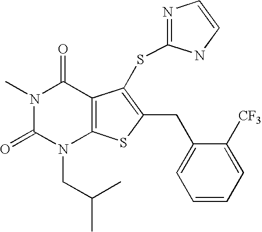

Prepared from the compound of Example 22.

m.p. 221-225° C.

MS (ES+) 495[M+H]

¹H NMR (CDCl₃) δ0.90 (6H,d), 2.14-2.23 (1H,m), 3.49 (3H,s), 3.69 (2H,d) 4.78 (2H,s), 7.11 (2H,d), 7.35-7.38 (2H,m), 7.43-7.48 (1H,m), 7.68 (1H,d), 11.34 (1H,br.s).

### EXAMPLE 32

3-Methyl-1-(2-methylpropyl)-5-(1,3,4-thiadiazol-2-ylthio)-6-[2-(trifluoromethyl)benzyl]-thieno[2,3-d]pyrimidine-2,4(1H,3H)dione

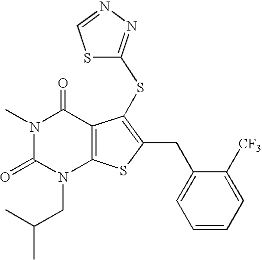

Prepared from the compound of Example 26.

m.p. 169-170° C.

MS (+ve APCI) 513 ((M+H)+)

¹H NMR (DMSO-D6) δ0.89 (6H, d), 2.08-2.12 (1H, m), 3.17 (3H, s), 3.71 (2H, d), 4.55 (2H, s), 7.40 (1H, d), 7.51 (1H, d), 7.64 (1H, t), 7.77 (1H, d), 9.45 (1H, s).

### EXAMPLE 33

5-[(5-Amino-1,3,4-thiadiazol-2-yl)thio]-3-methyl-1-(2-methylpropyl)-6-[2-(trifluoromethyl)benzyl]-thieno[2,3-d]pyrimidine-2,4(1H,3H)-dione

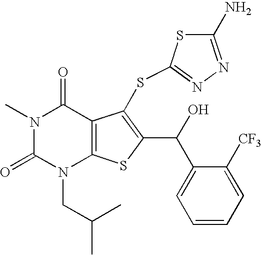

Prepared from the compounid of Example 25.

m.p. 236-7° C.

MS (+ve APCI) 528 ((M+H)+)

¹H NMR (DMSO-D6) δ0.86 (6H, d), 2.05-2.14 (1H, m), 3.23 (3H, s), 3.68 (2H, d), 4.56 (2H, s), 7.24 (2H, s), 7.37 (1H, d), 7.52 (1H, t), 7.64 (1H, t), 7.78 (1H,d).

### EXAMPLE 34

3-Methyl-1-(2-methylpropyl)-5-(2-thienylthio)-6-[[2-(trifluoromethyl)phenyl]methyl]-thieno[2,3-d]pyrimidine-2,4(1H,3H)-dione

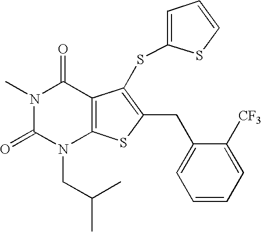

a) 6-[Hydroxy[2-(trifluoromethyl)phenyl]methyl]-3-methyl-1-(2-methylpropyl)-5(2-thienylthio)-thieno[2,3-d]pyrimidine-2,4(1H,3H)-dione

Prepared according to the method described in Example 27 using the compound of Example 24, and purified by flash silica chromatography eluting with a gradient of 10-30% ethyl acetate in isohexane.

MS(APCI) 508[M+H]+

b) 3-Methyl-1-(2-methylpropyl)-5-(2-thienylthio)-6-[[2-(trifluoromethyl)phenyl]methyl]-thieno[2,3-d]pyrimidine-2,4(1H,3H)-dione

Trifluoroacetic acid (4 ml) and triethylsilane (2 ml) were added to the product of step a) (0.11 g) and stirred at room temperature for 2 hours. The solution was concentrated under reduced pressure. The residue was basified and extracted with ethyl acetate. The combined extracts were dried over anhydrous magnesium sulfate, filtered and concentrated under reduced pressure. Purification by nornal phase HPLC eluting with a gradient of 5-60% ethyl acetate in isohexane gave a the title compound as a brown oil (0.011 g).

MS (ES+) 511[M+H]+

¹H NMR (CDCl₃) δ0.92 (6H,d), 2.04-2.24 (1H,m), 3.42 (3H,s), 4.11 (2H,d), 4.63 (2H,s), 6.91-6.94 (1H,m), 7.16 (1H,d), 7.28-7.47 (4H,m), 7.70 (1H,d).

Using the method of Example 14 the following compounds were prepared:

### EXAMPLE 35

3-Methyl-1-(2-methylpropyl)-5-[(5-methyl-1H-1,2,4-triazol-3-yl)sulfonyl]-6-[[2-(trifluoromethyl)phenyl]methyl]-thieno[2,3-d]pyrimidine-2,4(1H,3H)-dione

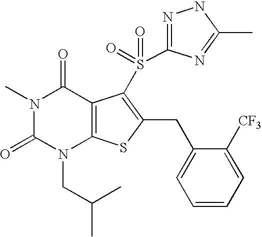

Prepared using the compound of Example 30 and purified by flash silica chromatography eluting with a gradient of 2-4% ethanol in dichloromethane.

MS(APCI) 542[M+H]+

¹H NMR (CDCl₃) δ0.92 (6H,d), 1 61 (1H,br.s.), 2.17 (1H,septet), 2.58 (3H,s), 3.28 (3H,s), 3.68 (2H,d), 5.02 (2H,s), 7.43-7.46 (lH,m), 7.53-7.60 (2H,m), 7.72 (1H,d).

### EXAMPLE 36

5-(1H-Imidazol-2-ylsulfonyl)-3-methyl-1-(2-methylpropyl)-6-[[2-(trifluoromethyl)phenyl]methyl]-thieno[2,3-d]pyrimidine-2,4(1H,3H)-dione

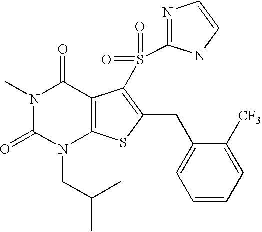

Prepared using the compound of Example 31 and purified by flash silica chromatography eluting with ethyl acetate:isohexane (1:1).

MS(APCI) 527[M+H]+

¹H NMR (CDCl₃) δ0.89 (6H,d), 2.12 (1H,septet), 3.40 (3H,s), 3.63 (2H,d), 5.03 (2H,s), 7.28-7.31 (2H,m), 7.40-7.57 (3H,m), 7.72 (1H,d), 11.74 (1H,br.s).

### EXAMPLE 37

6-[Hydroxy[2-(trifluoromethyl)phenyl]methyl]-3-methyl-1-(2-methylpropyl)-5-(1H-1,2,4-triazol-3-ylsulfonyl)-thieno[2,3-d]pyrimidine-2,4(1H,3H)-dione

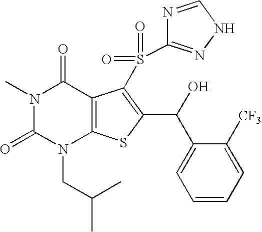

Prepared using the compound of Example 28 and purified by flash silica chromatography eluting with 4-10% ethanol in dichloromethane.

m.p. 198-202° C.

MS(APCI) 544[M+H]+

¹H NMR (CDCl₃+DMSO) δ0.90-0.97 (6H,mn), 2.20 (1H,septet), 2.57-2.60 (1H,m), 3.22 (3H,s), 3.65-3.82 (2H,m), 6.98 (1H,s), 7.48 (1H,t), 7.59 (1H,t), 7.74 (2H,d), 8.29 (1H,s).

### EXAMPLE 38

3-Methyl-1-(2-methylpropyl)-5-(1,3-thiazol-2-ylsunfonyl)-6-[2-(trifluoromethyl)benzyl]thieno[2,3-d]pyrimidine-2,4(1H,3H)-dione

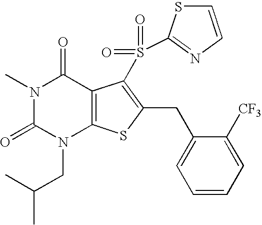

Prepared using the compound of Example 29 and purified by normal-phase preparative HPLC with gradient: dichloromethane/ethanol elution, followed by recrystallisation from ethyl acetate/iso-hexane.

MS (+ve APCI) 544 ((M+H)+)

¹H NMR (CDCl₃) δ0.91 (6H, d), 2.10-2.21 (1H, m), 3.27 (3H, s), 3.65 (2H, d), 5.03 (2H, s), 7.42-7.48 (1H, m), 7.53-7.59 (2H, m), 7.22-7.75 (2H, m), 7.91 (1H, d).

### EXAMPLE 39

3-Methyl-1-(2-methylpropl)-5-(1,3,4-thiadiazol-2-ylsulfonyl)-6-[2-(trifluoromethyl)benzyl]-thieno[2,3-d]pyrimidine-2,4(1H,3H)-dione

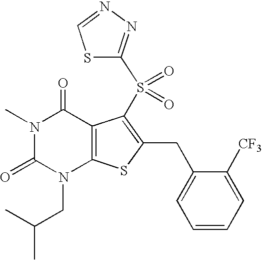

Prepared using the compound of Example 32 and purified by column chromatography over silica, eluting with ethyl acetate/isohexane (2:1), and then recrystallised from ethyl acetate/isohexane.

m.p. 148-149° C.

MS (+ve APCI) 545 ((M+H)+)

¹H NMR (DMSO-D6) δ0.85 (6H, d), 2.00-2.12 (1H, m), 3.08 (3H, s), 3.65 (2H, d), 4.96 (2H, s), 7.56 (1H, d), 7.59 (1H, t), 7.73 (1H, t), 7.82 (1H, d), 9.94 (1H, s).

### EXAMPLE 40

5-1(3-Bromo-1H-1,2,4-triazol-5-yl)thio]-3-methyl-1-(2-methylpropyl)-6-[2-(trifluoromethyl)phenylmethyl]-thieno[2,3-d]pyrimidine-2,4(1H,3H)-dione

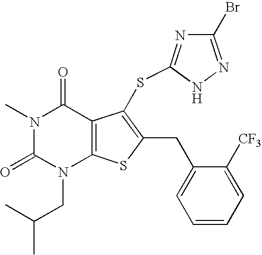

a) 3,5-Dibromo-1-(4-methoxyphenylmethyl)-1H-1,2,4-triazole

4-Methoxyphenylmethyl chloride (4.47 ml) was added to a stirred solution of 3,5-dibromo-1,2,4-triazole (7.125 g) and triethylamine (4.81 ml) in anhydrous dichloromethane (70 ml) at room temperature. After 4 days, the mixture was evaporated under reduced pressure and the residue purified by column chromatography over silica, eluting with ethyl acetate/isohexane (1:4) to give the sub-title compound (7.34 g) as a solid.

MS (+ve APCI) 346/348/350 ((M+H)+).

b) 5-{[3-Bromo-1-(4-methoxyphenylmethyl)-1H-1,2,4-triazol-5-yl]thio}-3-methyl-1-(2-methylpropyl)-6-[2-(trifluoromethyl)phenylmethyl]-thieno[2,3-d]yrimidine-2,4(1H,3H)-dione

A tetrahydrofuran solution of isopropylmagnesium chloride (2M, 0.63 ml) was added to a stirred solution of the product of Example 1, part b) (0.50 g) in anhydrous tetrahydrofuran (5 ml) at 0° C. under nitrogen. After 15 minutes, flowers of sulfur (0.04 g) was added and stirring continued at 0° C. for 30 minutes. Hydrochloric acid (1M, 20 ml) was added and the mixture extracted with ethyl acetate (2×20 ml). Organic extracts were dried over anhydrous magnesium sulfate, filtered and evaporated under reduced pressure. The residue was disssolved in tetrahydrofuran (5 ml) under nitrogen, saturated aqueous sodium bicarbonate solution (1 ml) was added followed by sodium borohydride (0.1 g) and the mixture stirred for 30 minutes. The product of step a) (0.44 g) was added and stirring continued for 18 hours at room temperature then for 30 minutes at reflux. Saturated aqueous sodium bicarbonate solution (20 ml) was added and the mixture extracted with ethyl acetate (2×20 ml). Organic extracts were dried over anhydrous magnesium sulfate, filtered and evaporated under reduced pressure. The residue was purified by column chromatography over silica, eluting with ethyl acetate/isohexane (1:4) to give the sub-title compound (0.368 g) as a solid.

MS (+ve APCI) 694/696 ((M+H)+).

c) 5-[(3-Bromo-1H-1,2,4-triazol-5-yl)thio]-3-methyl-1-(2-methylpropyl)-6-[2-(trifluoromethyl)phenylmethyl]-thieno[2,3-d]pyrimidine-2,4(1H,3H)-dione

A solution of the product of step b) (0.15 g) in anisole (2 ml) and trifluoroacetic acid (0.5 ml) was heated at reflux for 24 hours then evaporated under reduced pressure. The residue was purified by column chromatography over silica, eluting with ethyl acetate/isohexane (1:2) followed by recrystallisation from ethyl acetate/isoohexane to give the title-compound (0.075 g).

m.p. 174-5° C.

MS (+ve APCI) 574/576 ((M+H)+)

¹H NMR (CDCl₃ ) δ0.89 (6H, d), 2.08-2.17 (1H, m), 3.18 (3H, s), 3.71 (2H, d), 4.50 (2H, s), 7.38 (1H, d), 7.50 (1H, t), 7.62 (1H, t), 7.76 (lH, d).

### EXAMPLE 41

3-Methyl-1-(2-methylpropyl)-5-[(5-oxo-4,5-dilydro-1H-1,2,4-triazol-3-yl)thio]-6-[2-(trifluoromethyl)phenylmethyl]-thieno[2,3-d]pyrimidine-2,4(1H,3H)-dione

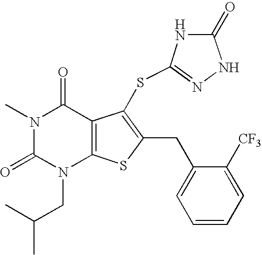

A solution of the product of Example 40, step b) ( 0.47 g) in trifluoroacetic acid (2 ml) and anisole (4 ml) was heated at 90° C. for 40 hours. The mixture was evaporated under reduced pressure and the residue purified by column cliromatograplhy over silica, eluting with ethyl acetate/isohexane (2:3 then 2:1) then ethyl acetate/methanol (19:1) to give the title compound (70 mg) which was recrystallised from ethyl acetate/isohexane.

m.p. 205-211° C.

MS (+ve APCI) 512 ((M+H)+)

¹H NMR (DMSO-D6) δ0.87 (6H, d), 2.06-2.16 (1H, m), 3.22 (3H, s), 3.69 (1H, d), 4.47 (2H, s), 7.36 (1H, d), 7.51 (1H, t), 7.62 (1H, t), 7.77 (1H, t), 11.43 (1H, s, br), 11.46 (1H, s, br).

### EXAMPLE 42

3-Methyl-1-(1-methylethyl)-5-(1,3-thiazol-2-ylthio)-6-[2-(trifluoromethyl)benzyl]-thieno[2,3-d]pyrimidine-2,4(1H,3H)-dione

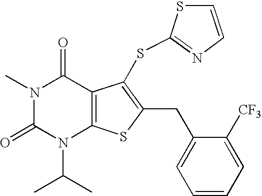

a) Ethyl 2-(isopropylamino)-3-thiophenecarboxylate

Triethylamine (80.5 ml) was added dropwise over 30 minutes to a stirred solution of 2,5-dihyhroxy-1,4-dithiane (43.9 g) and ethyl cyanoacetate (61.4 ml) in anhydrous dimethylfomiamide (200 ml) at 50° C. After a further 90 minutes the mixture was cooled to room temperature, added to water (1L) and extracted with dichloromethane (4×50 ml). Combined organic extracts were dried over anhydrous magnesium sulfate, filtered through a silica pad washing with ether (500 ml) and evaporated under reduced pressure. The residual oil was redissolved in anhydrous toluene (650 ml) then 2,2-dimethoxypropane (158 ml) and p-toluenesulphonic acid (1 g) were added. The mixture was heated under reflux under nitrogen for 5 hours then cooled to room temperature, added to saturated aqueous sodium bicarbonate solution (1L) and extracted with ether (1L). Organic extracts were dried over anhydrous sodium sulfate, filtered and evaporated under reduced pressure. The residual oil was redissolved in ethanol (250 ml) and treated with sodium borohydride (8 g). The mixture was stirred under nitrogen for 3 days then further sodium borohydride (8 g) was added and stirring continued for 1 day. Further sodium borohydride (4 g) was added and stirring continued for a further 2 days. Water (100 ml) was added followed by saturated aqueous sodium bicarbonate solution (750 ml) and the mixture extracted with ether (2×750 ml). Organic extracts were dried over anhydrous magnesium sulfate, filtered and evaporated under reduced pressure. The residue was purified by column chromatography over silica, eluting with isohexane/dichloromethane (1:1) to give the sub-title compound (24.7 g) as an oil.

MS (+ve APCI) 214 ((M+H)+)

¹H NMR (CDCl₃ ) δ1.31 (6H, d), 1.33 (3H, t), 3.47-3.53 (1H, m), 4.25 (2H, q), 6.14 (1H, d), 7.02 (1H, d), 7.36 (1H, s, br).

b) 3-Methyl-1-(1-methylethyl)-thieno[2,3-d]pyrimidine-2,4(1H,3H)-dione

Acetyl chloride (9.84 ml) was added dropwise to a stirred suspension of silver cyanate (21.6 g) in anhydrous toluene (100 ml) under nitrogen. After 30 minutes, a solution of the product of step a) (24.6 g) in anhydrous toluene (20 ml) was added. The mixture was stirred for a further 2 hours then diluted with ether (500 ml), filtered then washed with saturated aqueous sodium bicarbonate solution (200 ml). The aqueous layer was extracted with ether (200 ml) and combined organic extracts were dried over anhydrous magnesium sulfate, filtered and evaporated under reduced pressure. The residual oil was dissolved in ethanol (150 ml) and treated with sodium ethoxide (23.6 g). The mixture was stirred under nitrogen for 3 days then iodomethane (21.5 ml) was added. The mixture was heated under reflux for 3 hours then further iodomethane (10 ml) was added and heating continued for 1 hour. The mixture was cooled to room temperature, added to saturated aqueous sodium bicarbonate solution (1L) and extracted with ether (2×500 ml). Organic extracts were dried over anhydrous magnesium sulfate, filtered and evaporated under reduced pressure. The residue was purified by column chromatography over silica, eluting with isohexane/ether (1:1) to give the sub-title compound (15.8 g) as a solid.

MS (+ve APCI) 225 ((M+H)+)

¹H NMR (CDCl₃ ) δ1.59 (6H, d), 1.61 (3H, t), 3.41 (3H, s), 4.64-4.80 (1H, m), 6.85 (1H, d), 7.37 (1H, d).

c) 5-Bromo-6-{hydroxy[2-(trifluoromethyl)phenyl]methyl}-3-methyl-1-I(1-methylethyl)-thieno[2,3-d]pyrimidine-2,4(1H,3H)-dione

Bromine (1.38 ml) was added to a stirred solution of the product of step b) (5.0 g) in dichloromethane (100 ml). After 10 minutes, the solution was evaporated under reduced pressure. The residue was dissolved in ether (200 ml), washed with saturated aqueous sodium metabisulphite solution (100 ml) then saturated aqueous sodium bicarbonate solution (100 ml), dried over anhydrous magnesium sulfate, filtered and evaporated under reduced pressure. The residue was dissolved in anhydrous tetrahydrofuran (75 ml) under nitrogen and cooled to −78° C. A solution of lithium diisopropylamide (44.6 mmol) in anhydrous tetrahydrofuran (25 ml) was added, the mixture warmed briefly to room temperature then re-cooled to −78° C. and 2-(trifluoromethyl)benzaldehyde (5.88 ml) was added. After 2 hours, saturated aqueous sodium bicarbonate solution (100 ml) was added, the mixture warmed to room temperature then extracted with ether (2×100 ml). Organic extracts were dried over anhydrous magnesium sulfate, filtered and evaporated under reduced pressure. The residue was purified by column chromatography over silica, eluting with isohexane/ether (2:1) to give the sub-title compound (6.88 g) as a solid.

MS (+ve APCI) 477/479 ((M+H)+)

¹H NMR (CDCl₃ ) δ1.59 (6H, d), 2.72 (1H, d), 3.37 (3H, s), 4.6-4.50 (1H, m), 6.58 (1H, d), 7.48 (1H, d), 7.64 (1H, t), 7.72 (1H, d), 7.79 (1H, d).

d) 5-Bromo-3-methyl-1-(1-methylethyl)-6-[2-(trifluoromethyl)benzyl]-thieno[2,3-d]pyrimidine-2,4(1H,3H)-dione

Trifluoroacetic acid (8 ml) followed by triethylsilane (4 ml) were added to the product of step c) (3.8 g) and the mixture stirred for 1 hour then evaporated under reduced pressure. The residue was dissolved in ethyl acetate (100 ml), washed with saturated aqueous sodium bicarbonate solution (100 ml), dried over anhydrous magnesium sulfate, filtered and evaporated under reduced pressure. The residue was purified by column chromatography over silica, eluting with isohexane/ethyl acetate (2:1) to give the sub-title compound (2.44 g) as a solid.

MS (+ve APCI) 461/463 ((M+H)+)

¹H NMR (CDCl₃) δ1.55 (6H, d), 3.39 (3H, s), 4.33 (2H, s), 4.30-4.45 (1H, m), 7.30 (1H, d), 7.39 (1H, t), 7.50 (1H, t), 7.70 (1H, d).

e) 3-Methyl-1-(1-methylethyl)-5-(1,3-thiazol-2-ylthio)-6-[2-(trifluoromethyl)phenylmethyl]-thieno[2,3-d]pyrimidine-2,4(1H,3H)-dione

A solution of ethylmagnesium bromide in tetrahydrofuran (1M, 1.52 ml) was added dropwise to a stirred solution of the product of step d) (0.50 g) in anhydrous tetrahydrofuran (10 ml) at 0° C. under nitrogen. After 15 minutes, a solution of 2-(1,3- thiazol-2-yldithio)-1,3-thiazole ( 0.28 g) in anhydrous tetrahydrofuran (5 ml) was added and stirring continued at 0° C. for 1 hour. Saturated aqueous sodium bicarbonate solution (20 ml) was added and the mixture extracted with ethyl acetate (2×20 ml). Organic extracts were dried over anhydrous magnesium sulfate, filtered and evaporated under reduced pressure. The residue was purified by column chromatography over silica, eluting with isohexane/ethyl acetate (4:1) and then recrystallised from ethyl acetate/isohexane to give the title-conmpound (0.36 g).

MS (+ve ESI) 498 ((M+H)+)

¹H NMR (CDCl₃ ) δ1.56 (6H, d), 3.33 (3H, s), 4.30-4.50 (1H, m), 4.56 (2H, s), 7.22 (1H, d), 7.27 (1H, d), 7.37 (1H, t), 7.47 (3H, t), 7.66 (1H, d), 7.69 (1H, d).

### EXAMPLE 43

3-Methyl-1-(1-methylethyl)-5-(1,3-thiazol-2-ylsulfonyl)-6-[2-(trifluoromethyl)benzyl]thieno[2,3-d]pyrimidine-2,4(1H,3H)-dione

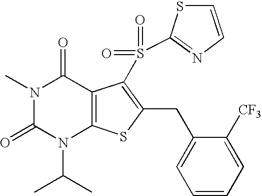

Prepared from the product of Example 29 by the method of Example 14 and purified by recrystallisation from ethyl acetate/isohexane.

MS (+ve APCI) 530 ((M+H)+)

¹H NMR (CDCl₃ ) δ1.51 (6H, d), 3.23 (3H, s), 4.22-4.62 (1H, m), 5.03 (2H, s), 7.42-7.48 (1H, m), 7.56-7.59 (2H, m), 7.71-7.75 (2H, m), 7.91 (1H, d).

### EXAMPLE 44

1-(Cyclopropylmethyl)-3-methyl-5-(2-thienylcarbonyl)-6-[2-(trifluoromethyl)phenylmethyl]-thieno[2,3-d]pyrimidine-2,4(1H,3H)-dione

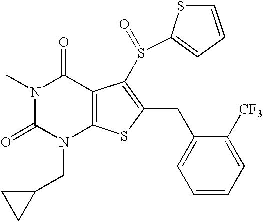

a) Diethyl 2-[(cyclopropylmethyl)amino]-3,4-thiophenedicarboxylate

Sodium borohydride (12.5 g) was added portionwise over 3 hours to a solution of diethyl 2-amino-3,4-thiophenedicarboxylate (25 g) in cyclopropanecarboxylic acid (125 ml). After a further 18 hours, further sodium borohydride (5 g) was added and the mixture heated at 50° C. for 18 hours. Further sodium borohydride (5 g) was added and the mixture stirred at 50° C. for 16 hours then at room temperature for 3 days. Water (500 ml) was added, the solution made alkaline by addition of sodium bicarbonate and extracted with ether (2×250 ml). Organic extracts were dried over anhydrous magnesium sulfate, filtered and evaporated under reduced pressure. The residue was purified by column chromatography over silica, eluting with ether/isohexane (2:3) to give the sub-title compound (12.1 g) as an oil.

MS (+ve APCI) 298 ((M+H)+)

b) Ethyl 1-(cyclopropylmethyl)-3-methyl-2,4-dioxo-1,2,3,4-tetrahydro-thieno[2,3-d]pyrimidine-5-carboxylate

Acetyl chloride (3.47 ml) was added to a stirred suspension of silver cyanate (7.63 g) in anhydrous toluene (20 ml). After 30 minutes, a solution of the product of step a) (12.1 g) in anhydrous toluene was added and the mixture stirred at room temperature under nitrogen for 16 hours. Ether (200 ml) was added and the mixture filtered. The precipitated solid was washed with saturated aqueous sodium bicarbonate solution (100 ml), and the combined filtrates were dried over anhydrous magnesium sulfate, filtered and evaporated under reduced pressure. The residual oil was dissolved in anhydrous ethanol (10 ml) and added to a stirred solution of sodium (2.81 g) in anhydrous ethanol (40 ml) at room temperature under nitrogen. After 4 hours, iodomethane (7.6 ml) was added, the mixture stirred at room temperature for 3 days then further iodomethane (3 ml) was added and the mixture heated at reflux for 3 hours. The mixture was cooled to room temperature and concentrated under reduced pressure to about 20 ml. Saturated aqueous sodium bicarbonate solution (200 ml) was added and the mixture extracted with ethyl acetate (2×200 ml). Organic extracts were dried over anhydrous magnesium sulfate, filtered and evaporated under reduced pressure to give the sub-title compound (11.8 g) as an oil.

MS (+ve APCI) 309 ((M+H)+)

c) Ethyl 1-(cyclopropylmethyl)-3-methyl-2,4-dioxo-6-[2-(trifluoromethyl)phenylmethyl]-1,2,3,4-tetrahydro-thieno[2,3-d]pyrimidine-5-carboxylate

The product of step b) (3.08 g) and 2-trifluoromethylbenzaldehyde (3.48 g) in dry tetrahydrofuran (50 ml) was cooled to −78° C. under nitrogen and treated with lithium diisopropylamide (2M, 11 ml). The reaction was held at −78° C. for 2 hrs and was then allowed to warm to −30° C., when water (3 ml) was added. The mixture was allowed to warm to room temperature and was thrown into water (500 ml), made slightly acidic with dil. HCl, and extracted with ethyl acetate. The organic solution was dried and evaporated and the residue was dissolved in dichloromethane (50 ml) and treated with triethylsilane (5 ml) and trifluoroacetic acid (10 ml). After 24 hr all volatiles were removed in vacuo and the residue was purified by column chromatography over silica, eluting with ether/isohexane (2:3) to give the sub-title compound (1.15 g) as a gum.

MS (+ve APCI) 467 ((M+H)+)

d) 1-(Cyclopropylmethyl)-3-methyl-2,4-dioxo-6-[2-(trifluorometlyl)benzyl]-1,2,3,4-tetrahydro-thieno[2,3-d]pyrimidine-5-carboxylic acid

The product of step c) (1.15 g) was treated with lithium hydroxide hydrate (240 mg) in water (6 ml) plus methanol (18 ml) and tetrahydrofuran (18 ml) at room temperature for 24 hr. The organic solvents were evaporated and the residue was diluted with water (25 ml) and acidified with dil. HCl. Extraction with ethyl acetate, drying and evaporation gave the sub-title compound (1.01 g) as a gum.

MS (+ve APCI) 439 ((M+H)+)

e) 1-(Cyclopropylmethyl)-3-methyl-2,4-dioxo-6-[2-1trifluoromethyl)phenylmethyl]-1,2,3,4-tetrahydro-thieno[2,3-d]pyrimidine-5-carbonyl chloride

Oxalyl chloride (0.40 ml) was added to a stirred solution of the product of step d) (1.00 g) and anhydrous dimethylfonnamide (0.1 ml) in anhydrous dichloromethane (20 ml) under nitrogen. After 1 hour, the solution was evaporated under reduced pressure to give the sub-title compound as a foam (1 g).

f) 1-(Cyclopropylmethyl)-3-methyl-5-(2-thienylcarbonyl)-6-[2-(trifluoromethyl)phenylmetkyl]-thieno[2,3-d]pyrimidine-2,4(1H,3H)-dione

A solution of the product of step e) (0.1 g) in anhydrous dichloromethane was added to a solution of thiophene (0.182 g) and tin(iv)chloride (0.01 ml) in dichloromethane (1 ml). After 30 minutes, sodium bicarbonate (0.1 g) was added, the mixture filtered and the filtrate applied to a silica column. Elution with an ethyl acetate/isohexane gradient followed by recrystallisation of the product from ethyl acetate/isohexane gave the title-compound (0.037 g).

m.p. 186-187° C.

MS (+ve APCI) 505 ((M+H)+)

¹H NMR (CDCl₃ ) δ0.44-0.48 (2H, m), 0.55-0.60 (2H, m), 1.21-1.29 (1H, m), 3.33 (3H, s), 3.81 (2H, d), 4.22 (1H, s), 7.12 (1H, t), 7.35 (1H, t), 7.44-7.50 (2H, m), 7.53 (1H,dd), 7.64 (1H, d), 7.73 (1H, dd).

### EXAMPLE 45

6-(9H-Fluoren-9-yl)-3-methyl-1-(2-methylpropyl)-5-(1H-1,2,4-triazol-5-ylsulfonyl)-thieno[2,3-d]pyrimidine-2,4(1H,3H)-dione

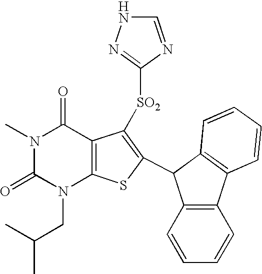

a) 5-Bromo-6-(9-hydroxy-9H-fluoren-9-yl)-3-methyl-1-(2-methylpropyl)-thieno[2,3-d]pyrimidine-2,4(1H,3H)-dione

Prepared by the method of Example 1, part a), using 9-fluorenone and purified by column chromatography over silica, eluting with isohexane/ethyl acetate (3:1) to give the sub-title compound as a solid.

MS (+ve APCI) 497/9 ((M+H)+)

b) 5-Bromo-6-(9H-fluoren-9-yl)-3-methyl-1-(2-methylpropyl)-thieno[2,3-d]pyrimidine-2,4(1H,3H)-dione

Prepared from the product of step a by the method of Example 1, part b) and purified by column chomatography over silica, eluting with dichloromethane/isohexane (3:1) to give the sub-title compound as a solid.

MS (+ve APCI) 481/3 ((M+H)+)

c) Bis[1-(4-methoxyphenylmethyl)-1H-1,2,4-triazol-5-yl]disulfide

4-Methoxyphenylmethyl chloride (7.77 g) was added to a stirred suspension of 1,2,4-thiadiazole-5(4H)-thione (2 g) and triethylamine (6.68 ml) in anhydrous tetrahydrofuran (50 ml). After 16 hours, water (50 ml) and ethyl acetate (50 ml) were added and the mixture acidified with 2M hydrochloric acid. Undissolved solid was collected by filtration, washed with water then ethyl acetate and dried in vacuo to give the sub-title compound (2.57 g).

MS (+ve APCI) 481 ((M+H)+)

d) 6-(9H-Fluoren-9-yl)-5-{[1-(4-methoxybenzyl)-1H-1,2,4-triazol-5-yl]thio}-3-methyl-1-(2-methylpropyl)-thieno[2,3-d]pyrimidine-2,4(1H,3H)-dione

Prepared from the products of steps b) and c) by the method of Example 42, part e) and purified by column chromatography over silica, eluting with isohexane/ethyl acetate (2:1) to give the sub-title compound as a solid.

MS (+ve ESI) 642 ((M+H)+).

e) 6-(9H-Fluoren-9-yl)-3-methyl-1-(2-methylpropyl)-5-(1H-1,2,4-triazol-5-ylsulfonyl)-thieno[2,3-d]pyrimidine-2,4(1H,3H)-dione

Aqueous hydrogen peroxide solution (30%, 0.5 ml) was added to a stirred suspension of the product of step d) (0.165 g) in acetic acid (1 ml) and dicliloromethane (1.5 ml). After 6 days, sodium hydroxide solution (2M, 10 ml) was added, the mixture stirred for 1 hour, diluted with water (25 ml), acidified with 2M hydrochloric acid and extracted with ethyl acetate (2×25 ml). Organic extracts were dried over anhydrous magnesium sulfate, filtered and evaporated under reduced pressure. The residue was purified by reverse-phase preparative HPLC with gradient aqueous ammonium acetate/acetonitrile elution and then further purified by column chromatography over silica, eluting with ethyl acetate/methanol (99:1) to give the title-compound (0.013 g) as a solid.

MS (+ve APCI) 534 ((M+H)+)

¹H NMR (CDCl₃ ) δ0.81 (6H, d), 1.96-2.05 (1H, m), 3.31 (3H, s), 3.54 (2H, d), 7.00 (1H, s), 7.37 (2H, t), 7.48 (2H, t), 7.76 (2H, d), 7.83 (2H, d), 8.49 (1H, s).

### EXAMPLE 46

6-(1H-Indol-3-ylcarbonyl)-3-methyl-1-(2-methylpropyl)-5-(1H-1,2,4-triazol-3-ylthio)-thieno[2,3-d]pyrirnidine-2,4(1H,3H)-dione

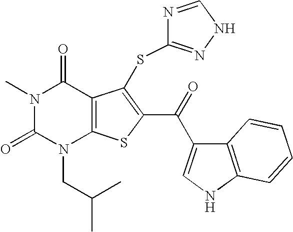

a) 5-Bromo-6-[hydroxy]1-[[2-(trimethylsilyl)ethoxy]methyl]-1H-indol-3-yl]methyl]-3-methyl-1-(2-methylpropyl)-thieno[2,3-d]pyrimidine-2,4(1H,3H)-dione

Prepared according to the method described in Example 1) part a) and purified using flash silica chromatography eluting with 20% ethyl acetate in isohexane to give the sub-title compound as a yellow foam.

MS(APCI) 606/608[M+H]+

b) 5-Bromo-3-methyl-1-(2-methylpropyl)-6-[[1-[[2-(trimetbylsilyl)ethoxy]methyl]1H-indol-3-yl]carbonyl]-thieno[2,3-d]pyrimidine-2,4(1H,3H)-dione

Prepared according to the method described in Example 12) part a) and purified by flash silica chromatography eluting with a gradient of 20-100% ethyl acetate in isohexane to give the sub-title compound as a pale green solid.

MS(APCI) 590/592[M+H]+

c) 3-Methyl-1-(2-methylpropyl)-5-(1H-1,2,4-triazol-3-ylthio)-6-[[1-[[2-(trimethylsilyl)ethoxy]methyl]-1H-indol-3-yl]carbonyl]-thieno[2,3-d]pyrimidine-2,4(1H,3H)-dione

Prepared according to the method described in Example 12) part b) and purified by flash silica chromatography eluting with 70% ethyl acetate in isohexane then 10% ethanol in dichloromethane to give the sub-title compound as a pale green solid.

MS(APCI) 611[M+H]+

d) 6-(1H-Indol-3-ylcarbonyl)-3-methyl-1-(2-methylpropyl)-5-(1H-1,2,4-triazol-3-ylthio)-thieno[2,3-d]pyrimidine-2,4(1H,3H)-dione

The product of step c) (0.25 g,), tetra-n-butylammonium fluoride (0.39 g) and ethylenediamine (0.122 ml) were dissolved in dry dimethylformamide (6 ml) and stirred at 80° C. under nitrogen for 20 hours. After cooling, the solution was partitioned between ethyl acetate and water. The organic layer was dried over anhydrous magnesium sulfate, filtered and concentrated under reduced pressure. The residue was purified by flash silica chromatography eluting with a gradient of 3-5% ethanol in dichloromethane to give the title compound as a yellow solid (0.081 g).

MS(APCI) 481[M+H]+

¹H NMR(CDCl₃) δ1.02 (6H,d), 2.34 (1H,septet), 3.33 (3H,s), 3.83 (2H,d), 7.25-7.27 (2H,m), 7.40-7.43 (1H,m), 7.71-7.72 (1H,br.in), 8.09 (1H,m), 8.24 (1H,br.m).

### EXAMPLE 47

6-(Hydroxy-(1H-indol-3-yl)methyl)-3-methyl-1-(2-methylpropyl)-5-(1H-1,2,4-triazol-3-ylthio)-thieno[2,3-d]pyrimidine-2,4(1H,3H)-dione

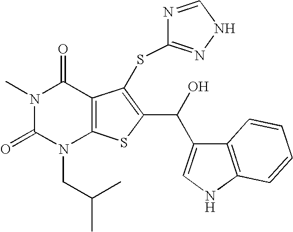

a) 6-[Hydroxy[1-[[2-(trimethylsilyl)ethoxy]methyl]-1H-indol-3-yl]methyl]-3-methyl-1-(2-methylpropyl)-5-(1H-1,2,4-triazol-3-ylthio)-thieno[2,3-d]pyrimidine-2,4(1H,3H)-dione

Prepared using the compound of Example 46 part c) according to the method described in Example 16 and purified by flash silica chromatography eluting with 40% acetone in isohexane to give the sub-title compound as a yellow foam.

MS(APCI) 595[M−H20]+

b) 6-(Hydroxy(1H-indol-3-yl)methyl)-3-methyl-1-(2-methylpropyl)-5-(1H-1,2,4-triazol-3-ylthio)-thieno[2,3-d]pyrimidine-2,4(1H,3H)-dione

Prepared using the product of step a) according to the method described in Example 46 part d) and purified by flash silica chromatography eluting with a gradient of 0-4% ethanol in dichloromethane to give the title compound as a brown solid.

MS(APCI) 465[M−H20]+

¹H NMR(CDCl₃) δ0.84-0.89 (6H,m), 2.13 (1H,septet), 3.40 (3H,s), 3.48 (1H,dd), 3.79 (1H,dd), 7.05 (2H,m), 7.09-7.10 (2H,m), 7.32-7.45 (3H,m), 8.04 (1H,s), 8.25 (1H,s).

### EXAMPLE 48

3-Methyl-1-(2-methylpropyl)-6-(4-quinolinylcarbonyl)-5-(1,3-thiazol-2-ylthio)-thieno[2,3-d]pyrimidine-2,4(1H,3H)-dione

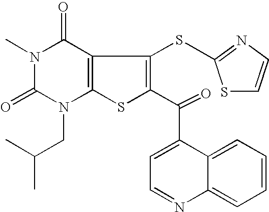

a) 5-Bromo-6-[hydroxy(4-quinolinyl)methyl]-3-methyl-1-(2-methylpropyl)-thieno[2,3-d]pyrimidine-2,4(1H,3H)-dione

Prepared by the method of Example 1 part a) and purified by column chromatography over silica eluting with isohexane/ethyl acetate (3/2) to give the sub-title compound.

MS (+ve APCI) 473 ((M+H)+)

¹H NMR (CDCl₃) δ9 (1H,d), 8.2 (1H, d), 7.9 (2H, in), 7.7 (1H, t), 7.5 (1H, t), 6.9 (1H,d), 3.4-3.8 (2H, dd), 3.4 (3H,s), 3.2 (1H,d), 2.1 (1H, m), 0.9 (3H,d), 0.9 (3H, d).

b) 5-Bromo-3-methyl-1-(2-methylpropyl)-6-(4-quinolinylcarbonyl)-thieno[2,3-d]pyrimidine-2,4(1H,3H)-dione

Prepared from the product of step a) by the method of Example 12 part a) and purified by passage through a.pad of silica eluting with dichloromethane to give the sub-title compound.

MS (+ve APCI) 473 ((M+H)+)

¹H NMR (CDCl₃) δ9 (1H,d), 8.2 (1H, d), 7.9 (2H, m), 7.6 (1H, t), 7.4 (1H, d) 3.9 (2H, d), 3.4 (3H,s), 3.2 (1H,d), 2.15 (1H, m), 1 (6H,d)

c) 3-Methyl-1-(2-methylpropyl)-6-(4-quinolinylcarbonyl)-5-(1,3-thiazol-2-ylthio)-thieno[2,3-d]pyrimidine-2,4(1H,3H)-dione

Prepared from the product of step b) by the method of Example 12 part b) and purified by column chromatography over silica, eluting with isohexane/ethyl acetate (1/1) then isohexane/ethyl acetate (2/3) followed by reciystallisation from ethyl acetate/iso-hexane to give the title compound (0.26 g).

m.p. 166.8-167.6° C.

MS (+ve APCI) 509 ((M+H)+)

¹H NMR (CDCl₃) δ1.04 (d, 6H), 2.3-2.4 (m, 1H), 3.3 (s,3H), 3.8 (d,2H), 7 (d,1H), 7.4 (d,1H), 7.49-7.55(,2H) 7.7 (t, 1H), 7.9 (d,1H), 8.1 (d,1H), 8.8 (d,1H)

### EXAMPLE 49

6-[Hydroxy-(4-quinolinyl)-methyl]-3-methyl-1-(2-methylpropyl)-5-(1,3-thiazol-2-ylthio)-thieno[2,3-d]pyrimidine-2,4(1H,3H)-dione

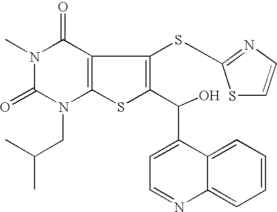

Prepared from the compound of Example 48 part c) by the method of Example 27 and purified using a reverse phase preparative HPLC with an isocratic gradient acetonitrile/0.1% ammonium aqueous solution (75/25) to give the title compound.

m.p. 182.8-184° C.

MS (+ve APCI) 511 ((M+H)+)

¹H NMR (CDCl₃) δ9.1 (1H,d), 8.2 (1H, d), 8.1 (1H, d), 7.8 (1H, d), 7.7 (2H, t), 7.5 (1H, t), 7.4 (1H, d), 7.4 (1H,d), 6.8 (1H,bs), 3.4-3.8 (2H, dd), 3.4 (3H,s), 2.1 (1H, m), 0.9 (3H,d), 0.9 (3H, d)

### EXAMPLE 50

6-(1-Benzothien3-ylmethyl)-3-methyl-1-(2-methylpropyl)-5-(4H-1,2,4-triazol-3-ylthio)-thieno[2,3-d]pyrimidine-2,4(1H,3H)-dione

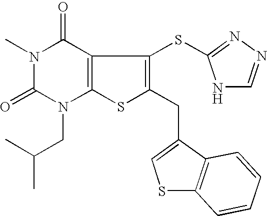

a) 6-[1-Benzothien-3-yl (hydroxy) methyl]-5-bromo-3-methyl-1-(2-mnethylpropyl)-thieno[2,3-d]pyrimidine-2,4(1H,3H)-dione

Prepared by the method of Example 1 part a) and purified by column chromatography over silica eluting with isohexane/ethyl acetate (2.3/1) to give the sub-title compound.

MS (+ve APCI) 481 ((M+H)+)

¹H NMR (CDCl₃) δ7.9 (1H, m), 7.7 (1H,m), 7.6 (1H, s), 7.4 (2H, m), 6.6 (1H, d) 3.5-3.9 (2H, dd), 3.4 (3H, s), 2.7 (1H, d), 2.1 (1H,m), 0.9 (3H,d), 0.9 (3H,d)

b) 6-(1-Benzothien-3-ylcarbonyl)-5-bromo-3-methyl-1-(2-methylpropyl)-thieno[2,3-d]pyrimidine-2,4(1H,3H)-dione

Prepared from the product of step a) by the method of Example 12 part a) and purified by passage through a pad of silica eluting with dichloromethane to give the title compound.

MS (+ve APCI) 479 ((M+H)+)

¹H NMR (CDCl₃) δ8.4 (1H, d), 8.2 (1H,s), 8.9 (1H, d), 7.5 (2H, m), 3.8 (2H, d), 3.4 (3H, s), 2.2 (1H,m), 1 (6H,d)

c) 5-(1-Benzothien-3-yl)-5-hydroxy-7-isobutyl-9-methyl-5H-pyrimido[5′,4′:4,5]thieno[2,3-e][1,2,4]triazolo[3,4-b][1,3]thiazine-8,10(7H,9H)-dione

Prepared from the product of step b) by the method of Example 12 part b) and purified by column chromatography over silica, eluting with isohexane/ethyl acetate (1/1) followed by recrystallisation from ethyl acetate/iso-hexanie to give the sub-title compound.

MS (+ve APCI) 498 ((M+H)+)

¹H NMR (CDCl₃) δ8.2 (1H, s), 7.8 (1H,s), 7.8 (1H, s), 7.1 (1H, t), 7.3 (1H,t), 7 (1H,bs), 6.8 (1H,d), 3.9−3.4 (2H, dd), 3.1 (3H, s), 2.1 (1H,m), 1 (3H,d), 1 (3H,d)

d) 6-(1-Benzothien-3-ylmethyl)-3-methyl-1-(2-methylpropyl)-5-(4H-1,2,4-triazol-3-ylthio)-thieno[2,3-d]pyrimidine-2,4(1H,3H)-dione

Prepared from the product of step c) by the method of Example 13 and was purified by chromatography over silica eluting with ethyl acetate followed by recrystallisation from dichloromethane/isohexane to give the title compound.

m.p. 237-238.2° C.

MS (+ve APCI) 484 ((M+H)+)

¹H NMR (CDCl₃) δ8 (1H, s), 7.9 (1H,m), 7.7 (1H, m), 7.35 (2H, m), 7.2 (1H,s), 4.7 (2H,s), 3.6 (2H, d), 3.4 (3H, s), 2.1 (1H,m), 0.9 (6H,d)

### EXAMPLE 51

6-(1H-Benzimidazol-1-ylmethyl)-3-methyl-1-(2-methylpropyl-5-(2-thienylthio)-thieno[2,3-d]pyrimidine-2,4(1H,3H)-dione

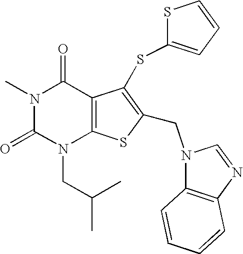

a) 6-Bromo-3-methyl-1-(2-methylpropyl)-thieno[2,3-d]pyrimidine-2,4(1H,3H)-dione

Bromine (2.79 ml) in dry dichloromethane (25 ml) was added dropwise to a solution of 3-methyl-1-(2-methylpropyl)-thieno[2,3-d]pyrimidine-2,4(1H,3H)-dione (12.0 g) in dry dichloromethane (100 ml) and stirred at room temperature for 30 minutes. The solution was concentrated under reduced pressure. The residue was dissolved in ethyl acetate and washed with sodium metabisulfite solution. The organic layer was dried over anhydrous magnesium sulfate, filtered and concentrated under reduced pressure to give the sub-title compound as a cream solid (15.85 g).

MS (APCI) 318 [M+H]+

b) 5-Bromo-3-methyl-1-(2-methylpropyl)-thieno[2,3-d]pyrimidine-2,4(1H,3H)-dione

To a solution of the product of step a) (5.30 g) in dry tetrahydrofuran (100 ml) at −78° C. was added lithium diisopropylamide (2M, 16.8 ml) dropwise over 10 minutes. After 90 minutes the reaction mixture was added to saturated ammonium chloride solution (100 ml) and left to warm to room temperature. The solution was extracted thrice with ethyl acetate, the resulting organic extracts were washed once with brine and then dried over magnesium sulfate, filtered and evaporated to leave the sub-title compound as a brown oil (4.00 g).

MS (+veAPCI) 317/319 ((M+H)+)

c) 5-Chloro-1,2,3,4-tetrahydro-3-methyl-1-(2-methylpropyl-2,4-dioxo-thieno[2,3-d]pyrimidine-6-carboxaldehyde

To a solution of the product of step b) (4.00 g) in phosphorus trichloride (80 ml) was added dimethylformarnide (1.5 ml) dropwise with stirring. The solution was heated for 18 hours at 100° C. under nitrogen. Once the solution had cooled to room temperature it was added dropwise to acidified warm water (1 ml of 2M hydrochloric acid in 500 ml of water at 50° C.) and then allowed to cool. The solution was extracted with ethyl acetate (3×200 ml). The organic phase was washed with brine and then dried over magnesium sulfate, filtered and evaporated to leave a dark brown oil. Colunn chromatography over silica eluting with dichloromethane, gave a solid which was triturated with diethyl ether to give the subtitle compound as a pale yellow solid (1.76 g).

¹H NMR (CDCl₃) δ1.00 (6H, d); 2.28-2.35 (1H, m); 3.42 (3H, s); 3.83 (2H,d); 10.10 (1H, s).

d) 1,2,3,4-Tetrahydro-3-methyl-1-(2-methylpropyl)-2,4-dioxo-5-(2-thienylthio)-thieno[2,3-d]pyrimidine-6-carboxaldehyde

To a solution of the product of step c) in acetone (30 ml) was added potassium carbonate (0.402 g) followed by 2-thiophenethiol (0.337 g). The reaction was stirred for 24 hours under nitrogen. The acetone was evaporated and the resulting residue was partitioned between ethyl acetate and water (1:1, 50 ml in total). The aqueous phase was extracted twice with ethyl acetate (25 ml). The organic extracts were washed with brine, then dried over magnesium sulfate, filtered and evaporated to leave a brown oil. This oil was triturated with diethyl ether to give the sub-title compound as a pale brown solid (0.528 g).

MS (+veAPCI) 381 ((M+H)+)

¹H NMR (DMSO d-6) δ0.92 (6H, d); 2.15-2.22 (1H, m); 3.24 (3H, s); 3.75 (2H, d); 7.19 (1H, dd); 7.61 (1H, dd); 7.89 (1H, dd); 8.98 (1H, s).

e) 6-(Hydroxymethyl)-3-methyl-1-(2-methylpropyl)-5-(2-thienylmethyl)-thieno[2,3-d]pyrimidine-2,4(1H,3H)-dione

Sodium borohydride (0.06 g) was added to the product of step d) (0.25 g) in methanol (25 ml) and the mixture was stirred for 24 hours. The reaction was diluted with water (30 ml) and was then extracted with ethyl acetate (3×50 ml). The organic phases were washed with brine and dried over magnesium sulfate, filtered and evaporated to leave a green oil. Purification by chromatography over silica (1:4 ethyl acetate: isohexane) gave the subtitle compound as a yellow foam (0.125 g).

MS (+veAPCI) 383((M+H)+)

¹H NMR (DMSO d-6) δ0.91 (6H, d); 2.15-2.22 (1H, m); 3.24 (3H, s); 3.73 (2H, d); 4.77 (2H, d); 5.98 (1H, t); 7.00 (1H, dd); 7.31 (1H, dd); 7.57 (1H, dd).

f) 6-(Chloromethyl)-3-methyl-1-(2-methylpropyl)-5-(2-thienylthio)-thieno[2,3-d]pyrimidine-2,4(1H,3H)-dione

Thionyl chloride (170 μl) was added to the product of step e) (0.45 g) in dry dichloromethane (20 ml) and the mixture was stirred for 45 minutes. The solvent was evaporated to give the subtitle compound as an orange oil.

MS(+veAPCI) 411 [M+CH₃CH₂OH]+

g) 6-(1-Benzimidazol-1-ylmethyl)-3-methyl-1-(2-methylpropyl)-5-(2-thienylthio)-thieno[2,3-d]pyrimidine-2,4(1H,3H)-dione

Benzimidazole (0.088 g) and sodium hydride (0.025 g) were added to the product of step f) (0.20 g) in dry dimethylfonnamide (20 ml) and the reaction was stirred for 24 hours at room temperature. Water was added to the reaction mixture, which was then extracted with ethyl acetate (3×50 ml). The combined organic phases were washed with brine and dried over magnesium sulfate, filtered and evaporated to leave a pale brown solid. Purification by chromatography (1:3 ethyl acetate: isohexane) gave the title compound as a white solid (0.104 g).

Melting point 198° C.

MS (+veAPCI) 483 ((M+H)+)

¹H NMR (DMSO d-6) δ0.86 (6H, d); 2.06-2.15 (1H, m); 3.27 (3H, s); 3.64 (2H, d); 5.93 (2H s); 6.99 (1H, m); 7.22 (2H, m); 7.38 (1H, d); 7.45 (1H, m); 7.53 (1H, d); 6.67 (1H, m); 8.28 (1H, s).

By the method of Example 51 step g) the following compounds were prepared:

### EXAMPLE 52

6-(1H-Indol-2-ylmethyl)-3-methyl-1-(2-methylpropyl)-5-(2-thienylthio)-thieno[2,3-d]pyrimidine-2,4(1H,3H)-dione

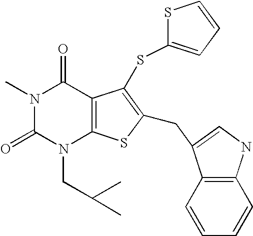

Melting point 156° C.

¹H NMR (CDCl₃) δ0.81 (6H, d), 2.12-2.24 (1H, m), 3.46 (3H, s), 3.63 (2H, d), 4.58 (2H, s), 6.94-6.99 (1H, m), 7.07-7.14 (2H, m), 7.19-7.34 (3H, m), 7.37-7.44 (2H,m), 7.46-7.53 (1H,m), 8.10-8.17 (1H,m).

### EXAMPLE 53

6-[(2-Chloro-6-methyl-1H-benzimidazol-1-yl)methyl]-3-methyl-1-(2-methylpropyl)-5-(2-thienylthio)-thieno[2,3-d]pyrimidine-2,4(1H,3H)-dione

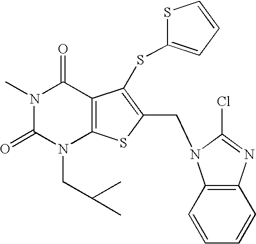

Meltng point 165° C.

¹H NMR (CDCl₃) δ0.81 (6H, d), 2.00-2.07 (1H, m), 3.22 (3H, s), 3.61 (2H, d), 5.97 (2H, s), 7.04 (1H, dd), 7.32 (2H, m), 7.41 (1H, d), 7.64 (3H, m),

### EXAMPLE 54

6-[(1,3-Dihydro-1,3-dioxo-2H-isoindol-2-yl)methyl]-3-methyl-1-(2-methylpropyl)-5-(2-thienylthio)-thieno[2,3-d]pyrimidine-2,4(1H,3H)-dione

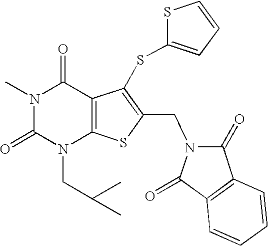

Melting point 163° C.

¹H NMR (CDCl₃) δ0.86 (6H, d), 2.09-2.16 (1H, m), 3.22 (3H, s), 3.68 (2H, d), 5.22 (2H, s), 6.99 (1H, dd), 7.37 (1H, d), 7.54 (1H, d), 7.89 (2H, dd), 7.93 (2H, dd).

### EXAMPLE 55

6-[Hydroxy[6-(trifluoromethyl)-2-pyridinyl]methyl]-3-methyl-1-(2-methylpropyl)-5-(2-thienylthio)-thieno[2,3-d]pyrimidine-2,4(1H,3H)-dione

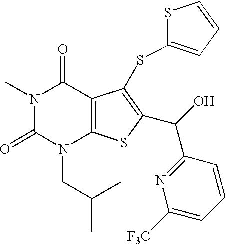

n-Butyl lithium (2.5M, 369 μl) was added dropwise to a solution of 2-bromo-6-trifluoromethylpyridine in tetrahydrofuran (8 ml) at −78° C. and the resulting solution was stirred for 1 hour. The product of Example 51 step d) (0.25 g) in tetrahydrofuran (2 ml) was added dropwise to the lithio pyridine solution. The reaction was stirred for 3 hours at −78° C. then saturated ammonium chloride solution was added (20 ml) and the reaction was left to warm to room temperature. The reaction was extracted thrice with ethyl acetate (50 ml), then the organic phases were washed with brine and dried over magnesium sulfate, filtered and evaporated to leave a brown oil. The residue was purified by chromatography over silica (1:4, ethyl acetate: isohexane) to give the title compound as a pale brown oil (0.03 g). p0 ¹H NMR (CDCl₃) δ0.90-0.99 (6H, m); 2.18-2.25 (1H, m); 3.58 (1H, dd); 3.80 (1H, dd); 4.55 (1H, d); 6.94-9.97 (1H, m); 7.10 (1H, d); 7.30-7.31 (1H, m); 7.43-7.45 (1H, m); 7.50-7.53 (1H, m); 8.06 (1H, d); 8.88 (1H, d).

### EXAMPLE 56

Inhibition of PMA/Ionomycin-Stimulated Peripheral Blood Mononuclear Cell Proliferation

The assay for PMA/ionomycin-stimulated PBMC proliferation was performed in 96-well flat bottomed microtitre plates. Compounds were prepared as 10 mM stock solutions in dimethyl sulfoxide. A 50-fold dilution of this was prepared in RPMI and serial dilutions were prepared from this solution. 10 μl of the 50-fold diluted stock, or dilutions of it, were added to the well to give concentrations in the assay starting at 9.5 μM and going down. Into each well was placed 1×10⁵ PBMC, prepared from human peripheral blood from a single donor, in RPMI1640 medium supplemented with 10% human serum, 2 mM glutamine and penicillin/streptomycin. Phorbol myristate acetate (PMA) (0.5 ng/ml final concentration) and ionomycin (500 ng/ml final concentration) were added to these cells in supplemented RPMI1640 medium (as above) so that the final volume of the assay was 0.2 ml. The cells were incubated at 37° C. in a humidified atmosphere at 5% carbon dioxide for 72 hours. ³H-Thyinidine (0.5 μCi) was added for the final 6 hours of the incubation. The level of radioactivity incorporated by the cells was then determined and this is a measure of proliferation.

The title compounds of Examples 1 to 11 were found to exhibit an IA₅₀ value of less than 1×10⁻6 M in the above test.

1. A compound of general formula 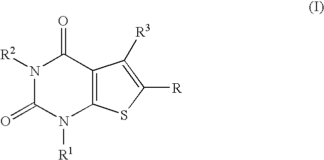
wherein:
R is —C(O)Ar¹, —C(R⁴)(R⁵)Ar¹ or Ar³;
Ar¹ represents a 5- to 10-membered aromatic ring system wherein up to 3 ring atoms may be heteroatoms independently selected from nitrogen, oxygen and sulphur, the ring system being optionally substituted by one or more substituents independently selected from C₁₋₄ alkyl, C₁₋₄ alkoxy, halogen, trifluoromethyl, oxo, nitro, cyano, NR⁶R⁷ and —CH₂NR⁸R⁹;
R¹ and R² each independently represent a hydrogen atom, C₁₋₆ alkyl, C₃₋₆ alkenyl, CH₂C₃₋₅ cycloalkyl or C₃₋₆ cycloalkyl;
R³ represents a group X—Ar²;
X represents a group S(O)ₙ, C(O) or CH(OH);
n is 0, 1 or 2;
Ar² represents a 5- or 6-membered aromatic ring wherein up to 4 ring atoms may be heteroatoms independently selected from nitrogen, oxygen and sulphur, the ring being optionally substituted by one or more substituents independently selected from C₁₋₄ alkyl, C₁₋₄ alkoxy, C₁₋₄ alkylthio, halogen, trifluoromethyl, oxo, hydroxyl, amino, nitro, cyano and benzyl;
R⁴ represents a hydrogen atom or C₁₋₄ allyl;
R⁵ represents a hydrogen atom or hydroxyl group;
R⁶ and R⁷ each independently represent a hydrogen atom or C₁₋₄ alkyl, or together with the nitrogen atom to which they are attached form a 5- to 7-membered saturated heterocyclic ring;
R⁸ and R⁹ each independently represent a hydrogen atom or C₁₋₄ alkyl, or together with the nitrogen atom to which they are attached fonn a 5- to 7-memnbered saturated heterocyclic ring; and
Ar³ represents acenaphthenyl, indanyl or fluorenyl, each of which may be optionally substituted by one or more substituents independently selected from C₁₋₄ alkyl, C₁₋₄ alkoxy, halogen or trifluoromethyl;
with the proviso that when X represents S(O)ₙ, then Ar² does not represent pyridyl or thieiiyl;
or a pharmaceutically acceptable salt or solvate thereof.

2. A compound according to claim 1, wherein R represents —C(R⁴)(R⁵)Ar¹.

3. A compound according to claim 1 or claim 2, wherein Ar¹ represents phenyl, naphthyl, pyrazolyl, thienyl, oxazolyl, imidazolyl, pyridinyl, pyridopyrrolyl, benzimidazolyl, indazolyl, benzothiazolyl, benzoxazolyl, thiazolyl or benzotriazolyl, each of which may be optionally substituted by one to four substituents independently selected from C₁₋₄ alkyl, C₁₋₄ alkoxy, halogen, trifluoromethyl, oxo, nitro, cyano, NR⁶R⁷ and —CH₂NR⁸R⁹.

4. A compound according to any one of claims 1 to 3, wherein R¹ and R² each independently represent a C₁₋₄ alkyl group.

5. A compound according to any one of claims 1 to 4, wherein Ar² represents furyl, pyridyl, thienyl, pyrrolyl, thiazolyl, thiadiazolyl, oxazolyl, imidazolyl, triazolyl or tetrazolyl, each of which may be optionally substituted by one to three substituents independently selected from C₁₋₄ alkyl, C₁₋₄ alkoxy, C₁₋₄ alkylthio, halogen, trifluoromethyl, oxo, hydroxyl, amino (NH₂), nitro, cyano and benzyl.

6. A compound of formula (I), or a pharmaceutically acceptable salt or solvate thereof, according to claim 1 being:
5-[3-Furyl(hydroxy)methyl]-1-isobutyl-3-methyl-6-[2-(trifluoromethyl)benzyl]thieno[2,3-d]pyrimidine-2,4(1H,3H)-dione,
5-[2-Furyl(hydroxy)methyl]-1-isobutyl-3-methyl-6-[2-(trifluoromethyl)benzyl]-thieno[2,3-d]pyrimidine-2,4(1H,3H)-dione,
5-[Hydroxy(5-methyl-2-thienyl)methyl]-1-isobutyl-3-methyl-6-[2-(trifluoromethyl)benzyl]-thieno[2,3-d]pyrimidine-2,4(1H,3H)-dione,
5-[Hydroxy(3-pyridinyl)methyl]-1-isobutyl-3-methyl-6-[2-(trifluoromethyl)benzyl]-thieno[2,3-d]pyrimidine-2,4( 1H,3H)-dione,
5-[Hydroxy(2-thienyl)methyl]-1-isobutyl-3-methyl-6-[2-(trifluoromethyl)benzyl]thieno[2,3-d]pyrimidine-2,4(1H,3H)-dione,
5-[Hydroxy(3-thienyl)methyl]-1-isobutyl-3-methyl-6-[2-(trifluoromethyl)benzyl]-thieno[2,3-d]pyrimidine-2,4(1H,3H)-dione,
1-Isobutyl-3-methyl-5-[(1-methyl-1H-pyrrol-2-yl)carbonyl]-6-[2-(trifluoromethyl)benzyl]-thieno[2,3-dupyrimidine-2,4(1H,3H)-dione,
1-Isobutyl-3-methyl-5-(3-thienylcarbonyl)-6-[2-(trifluoromethyl)benzyl-thieno[2,3-d]pyrimidine-2,4(1H,3H)-dione,
1-Isobutyl-3-methyl-5-[(3-methyl-2-pyridinyl)carbonyl]-6-[2-(trifluoromethyl)benzyl]-thieno[2,3-d]pyrimidine-2,4(1H,3H)-dione,
1-Isobutyl-3-methyl-5-(3-pyridinylcarbonyl)-6-[2-(trifluoromethyl)benzyl]-thieno[2,3-d]pyrimidine-2,4(1H,3H)-dione,
1-Isobutyl-3-methyl-5-(2-thienylcarbonyl)-6-[2-(trifluoromethyl)benzyl]-thieno[2,3-d]pyrimidine-2,4(1H,3H)-dione,
3-Methyl-1-(2-methylpropyl)-5-(1H- 1,2,4-triazol-3-ylthio)-6-[2-(trifluoromethyl)benzoyl]-thieno[2,3-d]pyrimidine-2,4(1H,3H)-dione,
3-Methyl-1-(2-methylpropyl)-5-(1H-1,2,4-triazol-3-ylthio)-6-[[2-(trifluoromethyl)phenyllmethyl]-thieno[2,3-d]pyrimidine-2,4(1H,3H)-dione,
3-Methyl-1-(2-methylpropyl)-5-(1H-1,2,4-triazol-3-ylsulfonyl)-6-[[2-(trifluoromethyl)phenyl]methyl]-thieno[2,3-d]pyrimidine-2,4(1H,3H)-dione,
3-Methyl-1-(2-methylpropyl)-5-(2-thiazolylthio)-6-[2-(trifluoromethyl)benzoyl]-thieno[2,3-d]pyrimidine-2,4(1H,3H)-dione,
3-Methyl-1-(2-methylpropyl)-5-[[5-(methylthio)-1,3,4-thiadiazol-2-yl]thio]-6-[2-(tiifluoromethyl)benzoyl]-thieno[2,3-d]pyrimidine-2,4(1H,3H)-dione,
3-Methyl-5-[(4-methyl-2-oxazolyl)thio]-1-(2-methylpropyl)-6-[2-(trifluoromethyl)benzoyl]-thieno[2,3-d]pyrimidine-2,4(1H,3H)-dione,
3-Methyl-1-(2-methylpropyl)-5-(5-methyl-1H-1,2,4-triazol-3-yl)thio]-6-[2-(trifluoromethyl)benzoyl]-thieno[2,3-d]pyrimidine-2,4(1H,3H)-dione,
3-Methyl-1-(2-methylpropyl)-5-[(5-hydroxy-4-(1-methylethyl)-1H-1,2,4-triazol-3-yl)thio]-6-[2-(trifluoromethyl)benzoyl]-thieno[2,3 -d]pyrimidine-2,4(1H,3H)-dione,
3-Methyl-1-(2-methylpropyl)-5-[( -methyl-1H-tetrazol-5-yl)thio]-6-[2-(trifluoromethyl)benzoyl]- thieno[2,3-d]pyrimidine-2,4(1H,3H)-dione,
3-Methyl-1-(2-methylpropyl)-5-[(5-methyl-1,3,4-thiadiazol-2-yl)thio]-6-[2-(trifluoromethyl)benzoyl]-thieno[2,3-d]pyrimidine-2,4(1H,3H)-dione,
5-(1H-Imidazol-2-ylthio)-3-methyl-1-(2-methylpropyl)-6-[2-(trifluoromethyl)benzoyl]-thieno[2,3 -d]pyrimidine-2,4(1H,3H)-dione,
5-[[4-(1,1-Dimethylethyl)-4H-1,2,4-triazol-3-yl]thio]-3-methyl-1-(2-methylpropyl)-6-[2-(trifluoromethyl)benzoyl]-thieno[2,3-d]pyrimidine-2,4(1H,3H)-dione,
3-Methyl-1-(2-methylpropyl)-5-(2-thienylthio)-6-[2-(trifluoromethyl)benzoyl]-thieno[2,3-d]pyrimidine-2,4(1H,3H)-dione,
5-[(5-Amino-1,3,4-thiadiazol-2-yl)thio]-3-methyl-1-(2-methylpropyl)-6-[2-(trifluoromethyl)benzoyl]-thieno[2,3 -d]pyrimidine-2,4(1H,3H)-dione,
3-Methyl-1-(2-methylpropyl)-5-(1,3,4-thiadiazol-2-ylthio)-6-[2-(trifluoromethyl)benzoyl]-thieno[2,3-d]pyrimidine-2,4(1H,3H)-dione,
6-[Hydroxy[2-(trifluoromethyl)phenyl]methyl]-5-(1H-inidazol-2-ylthio)-3-methyl-1-(2-methylpropyl)-thieno[2,3-d]pyrimidine-2,4(1H,3H)-dione,
6-[Hydroxy[2-(trifluoromethyl)phenyl]methyl]-3-methyl-1-(2-methylpropyl)-5-(1H-1,2,4-triazol-3-ylthio)-thieno[2,3-d]pyrimidine-2,4(1H,3H)-dione,
3-Methyl-1-(2-methylpropyl)-5-(2-thiazolylthio)-6-[[2-(trifluoromethyl)-phenyl]methyl]-thieno[2,3-d]pyrimidine-2,4(1H,3H)-dione,
3-Methyl-1-(2-methylpropyl)-5-[(5-methyl-1H-1,2,4-triazol-3-yl)thio]-6-[[2-(trifluoromethyl)phenylmethyl]-thieno[2,3-d]pyrimidine-2,4(1H,3H)-dione dihydrochloride,
5-(1H-Imidazol-2-ylthio)-3-methyl-1-(2-methylpropyl)-6-[[2-(trifluoromethyl)phenyl]methyl]-thieno[2,3-d]pyrimidine-2,4(1H,3H)-dione,
3-Methyl-1-(2-methylpropyl)-5-(1,3,4-thiadiazol-2-ylthio)-6-[2-(trifluoromethyl)benzyl]-thieno[2,3-d]pyrimidine-2,4(1H,3H)-dione,
5-[(5-Amino-1,3,4-thiadiazol-2-yl)thio]-3-methyl-1-(2-methylpropyl)-6-[2-(trifluoromethyl)benzyl]-thieno[2,3-d]pyrimidine-2,4(1H,3H)-dione,
3-Methyl-1-(2-methylpropyl)-5-(2-thienylthio)-6-[[2-(trifluorometlyl)phenyl]methyl]-thieno[2,3-d]pyrimidine-2,4(1H,3H)-dione,
3-Methyl-1-(2-methylpropyl)-5-[(5-methyl-1H-1,2,4-triazol-3-yl)sulfonyl]-6-[[2-(trifluoromethyl)phenyl]methyl]-thieno[2,3-d]pyrimidine-2,4(1H,3H)-dione,
5-(1H-Imidazol-2-ylsulfonyl)-3-methyl-1-(2-methylpropyl)-6-[[2-(trifluoromethyl)phenyl]methyl]-thieno[2,3-d]pyrimidine-2,4(1H,3H)-dione,
6-[Hydroxy[2-(trifluoromethyl)phenyl]methyl]-3-methyl-1-(2-methylpropyl)-5-(1H-1,2,4-triazol-3-ylsulfonyl)-thieno[2,3-d]pyrimidine-2,4(1H,3H)-dione,
3-Methyl-1-(2-methylpropyl)-5-(1,3-thiazol-2-ylsulfonyl)-6-[2-(trifluoromethyl)benzyl]-thieno[2,3-d]pyrimidine-2,4(1H,3H)-dione,
3-Methyl-1-(2-methylpropyl)-5-(1,3,4-thiadiazol-2-ylsulfonyl)-6-[2-(trifluoromethyl)benzyl]-thieno[2,3-d]pyrimidine-2,4(1H,3H)-dione,
5-[(3-Bromo-1H-1,2,4-triazol-5-yl)thio]-3-methyl-1-(2-methylpropyl)-6-[2-(trifluoromethyl)phenylmethyl]-thieno[2,3-d]pyrimidine-2,4(1H,3H)-dione,
3-Methyl-1-(2-methylpropyl)-5-[(5-oxo-4,5-dihydro-1H-1,2,4-triazol-3-yl)thio]-6-[2-(trifluoromethyl)phenylmethyl]-thieno[2,3-d]pyrimidine-2,4(1H,3H)-dione,
3-Methyl-1-(1-methylethyl)-5-(1,3-thiazol-2-ylthio)-6-[2-(trifluoromethyl)benzyl]-thieno[2,3-d]pyrimidine-2,4(1H,3H)-dione,
3-Methyl-1-(1-methylethyl)-5-(1,3-thiazol-2-ylsulfonyl)-6-[2-(trifluoromethyl)benzyl]thieno[2,3-d]pyrimidine-2,4(1H,3H)-dione,
1-(Cyclopropylmethyl)-3-methyl-5-(2-thienylcarbonyl)-6-[2-(trifluoromethyl)phenylmethyl]-thieno[2,3-d]pyrimidine-2,4(1H,3H)-dione,
6-(9H-Fluoren-9-yl)-3-methyl-1-(2-methylpropyl)-5-(1H-1,2,4-triazol-5-ylsulfonyl)-thieno[2,3-d]pyrimidine-2,4(1H,3H)-dione,
6-(1H-Indol-3-ylcarbonyl)-3-methyl-1-(2-methylpropyl)-5-(1H-1,2,4-triazol-3-ylthio)-thieno[2,3-d]pyrimidine-2,4(1H,3H)-dione,
6-(Hydroxy-(1H-indol-3-yl)methyl)-3-methyl-1-(2-methylpropyl)-5-(1H-1,2,4-triazol-3-ylthio)-thieno[2,3-d]pyrimidine-2,4(1H,3H)-dione,
3-Methyl-1-(2-methylpropyl)-6-(4-quinolinylcarbonyl)-5-(1,3-thiazol-2-ylthio)-thieno[2,3-d]pyrimidine-2,4(1H,3H)-dione,
6-[Hydroxy-(4-quinolinyl)-methyl]-3-methyl-1-(2-methylpropyl)-5-(1,3-thiazol-2-ylthio)-thieno[2,3-d]pyrimidine-2,4(1H,3H)-dione,
6-(1-Benzothien-3-ylmethyl)-3-methyl-1-(2-methylpropyl)-5-(4H-1,2,4-triazol-3-ylthio)-thieno[2,3-d]pyrimidine-2,4(1H,3H)-dione,
6-(1H-Benzimidazol-1-ylmethyl)-3-methyl-1-(2-methylpropyl)-5-(2-thienylthio)-thieno[2,3-d]pyrimidine-2,4(1H,3H)-dione,
6-(1H-Indol-2-ylmethyl)-3-methyl-1-(2-methylpropyl)-5-(2-thienylthio)-thieno[2,3-d]pyrimidine-2,4(1H,3H)-dione,
6-[(2-Chloro-6-methyl-1H-benzimidazol-1-yl)methyl]-3-methyl-1-(2-methylpropyl)-5-(2-thienylthio)-thieno[2,3-d]pyrimidine-2,4(1H,3H)-dione,
6-[(1,3-Dihydro-1,3-dioxo-2H-isoindol-2-yl)methyl]-3-methyl-1-(2-methylpropyl)-5-(2-thienylthio)-thieno(2,3-d]pyrimidine-2,4(1H,3H)-dione, or
6-[Hydroxy[6-(trifluoromethyl)-2-pyridinyl]methyl]-3-methyl-1-(2-methylpropyl)-5-(2-thienylthio)-thieno[2,3-d]pyrimidine-2,4(1H,3H)-dione.

7. A process for the preparation of a compound of formula (I) as defined in claim 1 which comprises,
(a) when R represents C(O)Ar¹ and X represents a sulphur atom, reacting a compound of general formula 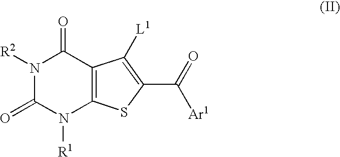
wherein L¹ represents a leaving group and R¹, R² and Ar¹ are as defined in formula (I), with a compound of general formula (III), HS—Ar², wherein Ar² is as defined in formula (I), in the presence of a base; or
(b) when R represents —C(R⁴)(R⁵)Ar¹, R⁴ represents a hydrogen atom, R⁵ represents hydroxyl and X represents a sulphur atom, reacting a corresponding compound of formula (I) in which R represents C(O)Ar¹ with a suitable reducing agent; or
(c) when R represents —C(R⁴)(R⁵)Ar¹, R⁴ represents a hydrogen atom, R⁵ represents a hydrogen atom and X represents a sulphur atom, reacting a corresponding compound of formula (I) in which R represents C(O)Ar¹ with a suitable reducing agent, in the presence of a Lewis acid; or
(d) when R represents —C(R⁴)(R⁵)Ar¹ and R⁴ represents C₁₋₄ alkyl, reacting a corresponding compound of formula (I) in which R represents C(O)Ar¹ with a suitable Grignard reagent, followed by reaction with a dehydrating agent in the presence of a base, and then followed by a hydrogenation reaction; or
(e) when X represents SO or SO₂, reacting a corresponding compound of formula (I) in which X is a sulphur atom with a suitable oxidising agent; or
(f) when X represents CH(OH), reacting a compound of general formula 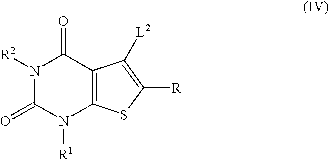
wherein L² represents a leaving group and R, R¹ and R² are as defined in formula (I), with a suitable Grignard reagent and then with a compound of general formula (V), Ar²CHO, wherein Ar² is as defined in formula (I); or
(g) when X represents C(O), reacting a corresponding compound of formula (I) in which X represents CH(OH) with a suitable oxidising agent; or
(h) when X represents a sulphur atom, reacting a compound of formula (IV) as defined in (f) above with a suitable alkyl lithium compound or Grignard reagent and then with a compound of general formula (VI), Ar²—S—S—Ar², wherein Ar² is as defined in formula (I);
and optionally after (a), (b), (c), (d), (e), (f), (g) or (h) forming a pharmaceutically acceptable salt or solvate of the compound of formula (I) obtained.

8. A pharmaceutical composition comprising a compound of formula (I) or a pharmaceutically acceptable salt or solvate thereof according to any one of claims 1 to 6 in association with a pharmaceutically acceptable adjuvant, diluent or carrier.

9. A process for the preparation of a pharmaceutical composition as claimed in claim 8 which comprises mixing a compound of formula (I) or a pharmaceutically acceptable salt or solvate thereof according to any one of claims 1 to 6 with a pharmaceutically acceptable adjuvant, diluent or carrier.

10. A compound of formula (I) or a pharmaceutically acceptable salt or solvate thereof as claimed in any one of claims 1 to 6 for use in therapy.

11. Use of a compound of formula (I) or a pharmaceutically acceptable salt or solvate thereof as claimed in any one of claims 1 to 6 in the manufacture of a medicament for use in therapy.

12. Use of a compound of formula (I) or a pharmaceutically acceptable salt or solvate thereof as claimed in any one of claims 1 to 6 in the manufacture of a medicament for use in the treatment of an obstructive airways disease.

13. Use according to claim 12, wherein the obstructive airways disease is asthma or chronic obstructive pulmonary disease.

14. Use of a compound of formula (I) or a pharmaceutically acceptable salt or solvate thereof as claimed in any one of claims 1 to 6 in the manufacture of a medicament for use in the treatment of allograft rejection.

15. A method of effecting immunosuppression which comprises administering to a patient a therapeutically effective amount of a compound of formula (I) or a pharmaceutically acceptable salt or solvate thereof as claimed in any one of claims 1 to 6.

16. A method of treating, or reducing the risk of, an obstructive airways disease in a patient suffering from, or at risk of, said disease, which comprises administering to the patient a therapeutically effective amount of a compound of formula (I) or a pharmaceutically acceptable salt or solvate thereof as claimed in any one of claims 1 to 6.
